(19) United States
(12) Patent Application Publication
(10) Pub. No.: US 2004/0121953 A1
(43) Pub. Date: Jun. 24, 2004

(54) Thrombopoiesis-stimulating proteins having reduced immunogenicity

(76) Inventors: Arthur J. Chirino, Camarillo, CA (US); Jamal El Yazal, Alta Loma, CA (US); Shannon Alicia Marshall, San Francisco, CA (US); Peter McDonnell, Thousand Oaks, CA (US); Jost Vielmetter, Altadena, CA (US)

Correspondence Address: Robin M. Silva, DORSEY & WHITNEY LLP, Suite 3400, Four Embarcadero Center, San Francisco, CA, 94111-4187, US

(21) Appl. No.: 10/638,995

(22) Filed: Aug. 11, 2003

Related U.S. Application Data
(60) Provisional application No. 60/467,609, filed on May 2, 2003.
(60) Provisional application No. 60/416,305, filed on Oct. 3, 2002.
(60) Provisional application No. 60/402,344, filed on Aug. 9, 2002.

(51) Int. Cl. C07K 14/575

(52) U.S. Cl. 514/12; 530/399

(57) ABSTRACT

The present invention relates to variant thrombopoietin proteins that possess thrombopoiesis-stimulating activity and have reduced immunogenicity. In particular, variants of thrombopoietin with reduced ability to bind one or more human class II MHC molecules are described.

This application is a continuation-in-part of U.S. Ser. No. 60/416,305, filed Oct. 3, 2002; U.S. Ser. No. 60/402,344, filed Aug. 9, 2002 and U.S. Ser. No. 60/467,609, filed May 2, 2003.

### FIELD OF THE INVENTION

The present invention relates to variant thrombopoietin proteins that possess thrombopoiesis-stimulating activity and have reduced immunogenicity. In particular, variants of thrombopoietin with reduced ability to bind one or more human class II MHC molecules are described.

### SEQUENCE LISTING

The Sequence Listing submitted on compact disc is hereby incorporated by reference. The two identical compact discs were created on Aug. 11, 2003 and contain the file named A71703-1.ST25.txt, created on Aug. 9, 2003, and containing 692,224 bytes.

### BACKGROUND OF THE INVENTION

Immunogenicity is a major barrier to the development and utilization of protein therapeutics. Although immune responses are typically most severe for non-human proteins, even therapeutics based on human proteins may be immunogenic. Immunogenicity is a complex series of responses to a substance that is perceived as foreign and can include production of neutralizing and non-neutralizing antibodies, formation of immune complexes, complement activation, mast cell activation, inflammation, and anaphylaxis.

Several factors can contribute to protein immunogenicity, including but not limited to the protein sequence, the route and frequency of administration, and the patient population.

Immunogenicity may limit the efficacy and safety of a protein therapeutic in multiple ways. Efficacy can be reduced directly by the formation of neutralizing antibodies. Efficacy can also be reduced indirectly, as binding to either neutralizing or non-neutralizing antibodies typically leads to rapid clearance from serum. Severe side effects and even death can occur when an immune reaction is raised. One special class of side effects results when neutralizing antibodies cross-react with an endogenous protein and block its function.

Thrombopoietin (TPO), also called mpl ligand or megakaryocyte growth and differentiation factor (MGDF), is a protein that acts to promote the growth and differentiation of platelets and other hematopoietic lineages (see for example Bartley et. al., Cell 77: 1117-1124 (1994), de Sauvage et. al. Nature 369: 533-538 (1994), Foster et. al. PNAS 91: 13023-13027(1994)). Therapeutic use of TPO could be beneficial in a wide range of conditions that result in inadequate platelet counts (e.g., thrombocytopenia) (See, for example, U.S. Pat. Nos. 5,989,537; 6,099,830; 5,766,581; 5,795,569; 5,326,558; and 5,593,666).

However, harmful immune reactions have occurred when patients have been treated with TPO. For example, clinical trials were terminated when healthy volunteers raised anti-TPO antibodies that cross-reacted with and neutralized endogenous TPO. Since these patients effectively lacked functioning TPO, they could not produce sufficient platelets and thus became thrombocytopenic (see for example Li et. al. Blood 96: 3241-3248 (2001)).

Several methods have been developed to modulate the immunogenicity of proteins. In some cases, PEGylation has been observed to reduce the fraction of patients who raise neutralizing antibodies by sterically blocking access to antibody epitopes (see for example, Hershfield et. al. PNAS 1991 88:7185-7189 (1991); Bailon. et al. Bioconjug. Chem. 12: 195-202(2001); He et a. Life Sci. 65: 355-368 (1999)). Methods that improve the solution properties of a protein therapeutic may also reduce immunogenicity, as aggregates have been observed to be more immunogenic than soluble proteins.

A more general approach to immunogenicity reduction involves mutagenesis targeted at the epitopes in the protein sequence and structure that are most responsible for stimulating the immune system. Some success has been achieved by randomly replacing surface-exposed residues to lower binding affinity to panels of known neutralizing antibodies (See, for example, U.S. Pat. No. 5,766,898). However, due to the incredible diversity of the antibody repertoire, mutations that lower affinity to known antibodies will most likely lead to production of an another set of antibodies rather than abrogation of immunogenicity.

An alternate approach is to disrupt T-cell activation. Removal of MHC-binding epitopes offers a much more tractable approach to immunogenicity reduction, as the diversity of MHC molecules comprises only ˜10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such epitopes for the purpose of generating less immunogenic proteins has been disclosed previously. See, for example, WO 98/52976, WO 02/079232, and WO 00/3317.

Methods for identifying and modifying MHC-binding epitopes in human TPO have been disclosed previously (See, for example, WO00/234,779 and WO02/068469). However, due to the large number of variants disclosed, and the lack of consideration of the structural and functional effects of the introduced mutations, one skilled in the art faces a problem in identifying a variant that would be a functional, non-immunogenic TPO variant suitable for administration to patients.

While mutations in MHC-binding epitopes can be identified that are predicted to confer reduced immunogenicity, most amino acid substitutions are energetically unfavorable. As a result, the vast majority of the reduced immunogenicity sequences identified using the methods described above will be incompatible with the structure and/or function of the protein. In order for MHC epitope removal to be a viable approach for reducing immunogenicity, it is crucial that simultaneous efforts are made to maintain a protein's structure, stability, and biological activity. Accordingly, there is a need to identify less immunogenic variants of TPO that significantly retain its desired thrombopoiesis-stimulating activity.

### SUMMARY OF THE INVENTION

The present invention relates to variants of TPO that substantially retain thrombopoiesis-stimulating activity and reduce or substantially eliminate immunogenicity relative to native TPO.

An aspect of the present invention are TPO variants that comprise peptides with decreased binding affinity for one or more class II MHC alleles relative to native human TPO and which significantly maintain the thrombopoiesis-stimulating activity of native human TPO.

In a further aspect, the invention provides recombinant nucleic acids encoding the variant TPO proteins as well as expression vectors and host cells encoding and producing variant TPO proteins.

An aspect of the present invention are TPO variants that show decreased binding affinity for one or more class II MHC alleles relative to native human TPO and which significantly maintain the thrombopoiesis-stimulating activity of native human TPO.

In a further aspect, the invention provides recombinant nucleic acids encoding the variant TPO proteins, expression vectors, and host cells.

In an additional aspect, the invention provides methods of producing a variant TPO protein comprising culturing the host cells of the invention under conditions suitable for expression of the variant TPO protein.

In a further aspect, the invention provides pharmaceutical compositions comprising a variant TPO protein of the invention and a pharmaceutical carrier.

In a further aspect, the invention provides methods for preventing or treating disorders related to insufficient platelet counts comprising administering a variant TPO protein of the invention to a patient.

In accordance with the objects outlined above, the present invention provides TPO variant proteins comprising amino acid sequences with at least one amino acid change compared to the wild type TPO proteins.

### BRIEF DESCRIPTION OF THE FIGURES

FIG. 1a shows the amino acid sequence of the N-terminal cytokine domain of human TPO (SEQ ID NO:1).

FIG. 1b shows the full-length amino acid sequence of human TPO (SEQ ID NO:2). GenBank gi|730982|sp|P402251|TPO_HUMAN Thrombopoietin precursor (Megakaryocyte colony stimulating factor) (Myeloproliferative leukemia virus oncogene ligand) (C-mpl ligand) (ML) (Megakaryocyte growth and development factor) (MGDF).

FIG. 2 shows one possible sequence alignment of human erythropoietin (EPO) (SEQ ID NO:3) and the N-terminal cytokine domain of TPO (SEQ ID NO:1).

FIG. 3 shows the number of DR alleles that are hit at 1%, 3%, and 5% thresholds by each 9-mer in wild type TPO.

FIG. 4 shows the results of Library 1 and epitope 1 BaF3 cell proliferation in the presence of various thrombopoietin variants. Wild type thrombopoietin (wt tpo) contains amino acid 1 to 157. Variants were derived from wt tpo, expressed in 293T cells, and the culture supernatant used to test activity. Commercial thrombopoietin was produced in E. coli and has 174 amino acid residues.

### DETAILED DESCRIPTION OF THE INVENTION

Definitions:

By “9-mer peptide frame” and grammatical equivalents herein is meant a linear sequence of nine amino acids that is located in a protein of interest. 9-mer frames may be analyzed for their propensity to bind one or more Class II MHC alleles.

By “allele” and grammatical equivalents herein is meant an alternative form of a gene. Specifically, in the context of Class II MHC molecules, alleles comprise all naturally occurring sequence variants of DRA, DRB1, DRB3/4/5, DQA1, DQB1, DPA1, and DPB1 molecules.

By “control sequences” and grammatical equivalents herein is meant nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

By “hit” and grammatical equivalents herein is meant, in the context of the matrix method, that a given peptide is predicted to bind to a given Class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity.

By “immunog nicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis.

By “matrix method” and grammatical equivalents thereof herein is meant a method for calculating peptide-MHC affinity in which a matrix is used that contains a score for each possible residue at each position in the peptide that interacts with a given MHC allele. The binding score for a given peptide is obtained by summing the matrix values for the amino acids observed at each position in the peptide.

By “MHC-binding epitopes” and grammatical equivalents herein is meant peptides that are capable of binding to one or more Class II MHC alleles with appropriate affinity to enable the formation of a MHC-peptide-T-cell receptor complex and subsequent T-cell activation. MHC-binding epitopes are linear peptides that comprise at least approximately 9 residues.

By “naturally occurring” or “wild-type” and grammatical equivalents thereof herein is meant an amino acid sequence or a nucleotide sequence that is found in nature and includes allelic variations. In a preferred embodiment, the wild-type sequence is the most prevalent human sequence. However, the wild type TPO proteins may be from any number of organisms, include, but are not limited to, rodents (rats, mice, hamsters, guinea pigs, etc.), primates, and farm animals (including sheep, goats, pigs, cows, horses, etc). As will be appreciated by those in the art, TPO proteins from mammals other than humans may find use in animal models of human disease.

By “nucleic acid” and grammatical equivalents herein is meant DNA, RNA, or molecules which contain both deoxy- and ribonucleotides. Nucleic acids include genomic DNA, cDNA and oligonucleotides including sense and anti-sense nucleic acids. Nucleic acids may also contain modifications, such as modifications in the ribose-phosphate backbone that confer increased stability and half-life. Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, elements such as enhancers do not have to be contiguous.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

“Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

By “protein” herein is meant a molecule comprising at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)] or non-canonical amino acids such as homo-phenylalanine, citrulline, hydroxyproline, and noreleucine. Both D- and L-amino acids may be utilized.

By “reduced immunogenicity” and grammatical equivalents herein is meant a decreased ability to activate the immune system, when compared to the wild type protein. For example, a TPO variant protein can be said to have “reduced immunogenicity” if it elicits neutralizing or non-neutralizing antibodies in lower titer or in fewer patients than wild type TPO. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A TPO variant protein also can be said to have “reduced immunogenicity” if it shows decreased binding to one or more MHC alleles or if it induces T-cell activation in a decreased fraction of patients relative to wild type TPO. In a preferred embodiment, the probability of T-cell activation is decreased by at least 5%, with at least 50% or 90% decreases being especially preferred.

By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 5 μg/kg are used, administered either intravenously or subcutaneously. As is known in the art, adjustments for variant TPO protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

“TPO-responsive disorders” and grammatical equivalents herein is meant diseases, disorders, and conditions that can benefit from treatment with TPO. Examples of TPO-responsive disorders include, but are not limited to, myelodysplastic syndromes, liver disease, amylotrophic lateral sclerosis, and immune thrombocytopenia purpura. TPO may also be beneficial for patients receiving treatments, such as for cancer, HIV, hepatitis C, and other diseases, that can result in low levels of platelets and other types of blood cells. TPO may also be beneficial for patients undergoing surgery or bone marrow transplantation. TPO may also be used to increase platelet concentrations in platelet donors.

By “treatment” herein is meant to include therapeutic treatment, as well as prophylactic, or suppressive measures for the disease or disorder. Thus, for example, successful administration of a variant TPO protein prior to onset of the disease may result in treatment of the disease. As another example, successful administration of a variant TPO protein after clinical manifestation of the disease to combat the symptoms of the disease comprises “treatment” of the disease. “Treatment” also encompasses administration of a variant TPO protein after the appearance of the disease in order to eradicate the disease. Successful administration of an agent after onset and after clinical symptoms have developed, with possible abatement of clinical symptoms and perhaps amelioration of the disease, further comprises “treatment” of the disease.

By “variant TPO nucleic acids” and grammatical equivalents herein is meant nucleic acids that encode variant TPO proteins. Due to the degeneracy of the genetic code, an extremely large number of nucleic acids may be made, all of which encode the variant TPO proteins of the present invention, by simply modifying the sequence of one or more codons in a way, which does not change the amino acid sequence of the variant TPO.

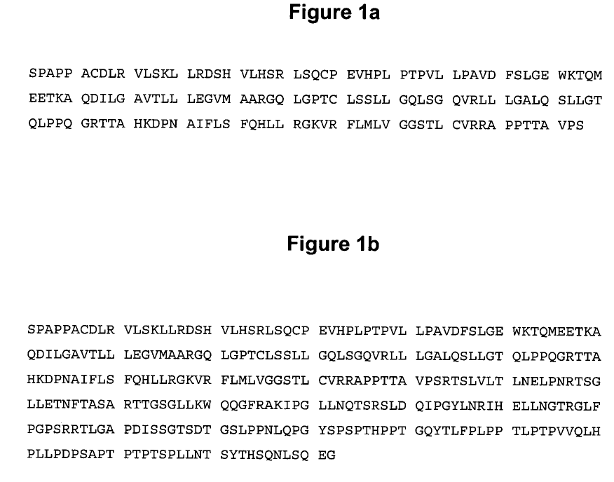

By “variant TPO proteins” or “non-naturally occurring TPO proteins” and grammatical equivalents thereof herein is meant non-naturally occurring TPO proteins which differ from the wild type TPO protein by at least one (1) amino acid insertion, deletion, or substitution. It should be noted that unless otherwise stated, all positional numbering of variant TPO proteins and variant TPO nucleic acids is based on these sequences. TPO variants are characterized by the predetermined nature of the variation, a feature that sets them apart from naturally occurring allelic or interspecies variation of the TPO protein sequence. The TPO variants typically either exhibit the same qualitative biological activity as the naturally occurring TPO or have been specifically engineered to have alternate biological properties. Variant TPO proteins comprise the N-terminal cytokine domain and may optionally include part or all of the C-terminal domain. In a preferred embodiment, the variant TPO comprises residues 1-153, 1-159, or 1-163 as shown in FIG. 1. The variant TPO proteins may contain insertions, deletions, and/or substitutions at the N-terminus, C-terminus, or internally. In a preferred embodiment, variant TPO proteins have at least 1 residue that differs from the human TPO sequence, with at least 2, 3, 4, or 5 different residues being more preferred. Variant TPO proteins may contain further modifications, for instance mutations that alter stability or solubility or which enable or prevent posttranslational modifications such as PEGylation or glycosylation. Variant TPO proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels.

Identification of MHC-Binding Epitopes in TPO

MHC-binding peptides are obtained from proteins such as TPO by a process called antigen processing. First, the protein is transported into an antigen presenting cell by endocytosis or phagocytosis. A variety of proteolytic enzymes then cleave the protein into a number of peptides. Next, these peptides can then be loaded onto Class II MHC molecules, and the resulting peptide-MHC complexes are transported to the cell surface. Relatively stable peptide-MHC complexes can be recognized by T-cell receptors that are present on the surface of naive T-cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the Class II MHC molecule. While peptides that bind Class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding epitopes in protein sequences are known in the art and may be used to identify epitopes in TPO.

Sequence-based information can be used to determine a binding score for a given peptide-MHC interaction (see for example Brusic et. al. Bioinformatics 14: 121-130 (1998) Reche et. al. Hum. Immunol. 63: 701-709 (2002); Mallios, Bioinformatics 15: 432-439 (1999); Mallios, Bioinformatics 17: p942-948 (2001); Sturniolo et. al. Nature Biotech. 17: 555-561(1999)). It is possible to use structure-based methods in which a given peptide is computationally placed in the peptide-binding groove of a given MHC molecule and the interaction energy is determined (See, for example, Altuvia et. al. J. Mol. Biol. 249:244-250 (1995), WO98/59244 and WO02/069232). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (See, for example, WO 02/77187).

In a preferred embodiment, MHC-binding propensity scores are calculated for each 9-residue frame along the human TPO sequence using a matrix method (see Sturniolo et. al., supra; Marshall et. al., J. Immunol. 154: 5927-5933 (1995), and Hammer et. al., J. Exp. Med. 180: 2353-2358 (1994)). The matrix comprises binding scores for specific amino acids interacting with the peptide binding pockets in different human Class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 mM or 10 mM or 1 mM affinity.

In a preferred embodiment, the number of hits for each 9-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding epitopes are identified as the 9-mer frames that bind to several Class II MHC alleles. In an especially preferred embodiment, MHC-binding epitopes are predicted to bind at least 10 alleles at 5% threshold and/or at least 5 alleles at 1% threshold. Such 9-mer frames may be especially likely to elicit an immune response in many members of the human population.

In an alternate preferred embodiment, MHC-binding epitopes are predicted to bind MHC alleles that are present in at least 0.01-10% of the human population.

In an additional preferred embodiment, MHC-binding epitopes are identified as the 9-mer frames that are located among “nested” epitopes, or overlapping 9-residue frames that are each predicted to bind a significant number of alleles. Such sequences may be especially likely to elicit an immune response.

Preferred MHC-binding epitopes in TPO include, but are not limited to, residues 9-17,11-19,15-23, 16-24, 69-77, 97-105, 135-143, 139-147, 144-152, 152-160, 296-304, and 297-305.

Especially preferred MHC-binding epitopes in native TPO include, but are not limited to, residues 9-17 and 135-143. These epitopes are both predicted to bind to a large number of MHC alleles. Furthermore, these epitopes are located within regions of nested epitopes; for example, residues 9-17 overlap with the immunogenic epitopes at residues 11-19, 15-23, and 16-24.

Confirmation of MHC-Binding Epitopes

In a preferred embodiment, the immunogenicity of the above-predicted MHC-binding epitopes is experimentally confirmed by measuring the extent to which peptides comprising each predicted epitope can elicit an immune response. However, it is possible to proceed from epitope prediction to epitope removal without the intermediate step of epitope confirmation.

Several methods, discussed in more detail below, can be used for experimental confirmation of epitopes. For example, sets of naive T-cells and antigen presenting cells from matched donors can be stimulated with a peptide or protein containing an epitope of interest, and T-cell activation can be monitored. In a preferred embodiment, interferon gamma production by activated T-cells is monitored, although it is also possible to use other indicators of T-cell activation or proliferation such as tritiated thymidine incorporation or interleukin 5 production.

Design of Active, Less-Immunogenic Variants

In a preferred embodiment, the above-determined MHC-binding epitopes are replaced with alternate amino acid sequences to generate active variant TPO proteins with reduced or eliminated immunogenicity. Alternatively, the MHC-binding epitopes are modified to introduce one or more sites that are susceptible to cleavage during protein processing. If the epitope is cleaved before it binds to a MHC molecule, it will be unable to promote an immune response. There are several possible strategies for integrating methods for identifying less immunogenic sequences with methods for identifying structured and active sequences, including but not limited to those presented below.

Protein design methods and MHC epitope identification methods can be used together to identify stable, active, and minimally immunogenic protein sequences (see WO03/006154). The combination of approaches provides significant advantages over the prior art for immunogenicity reduction, as most of the reduced immunogenicity sequences identified using other techniques fail to retain sufficient activity and stability to serve as therapeutics.

A wide variety of methods are known for generating and evaluating sequences. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)).

In a preferred embodiment, rational design of novel TPO variants is achieved by using Protein Design Automation® (PDA®) technology. (See U.S. Pat. Nos. 6,188,965; 6,269,312; 6,403,312; WO98/47089 and U.S. Ser Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all references expressly incorporated herein in their entirety.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization

PDA® utilizes three-dimensional structural information. In the most preferred embodiment, the structure of TPO is obtained by solving its crystal structure or NMR structure by techniques well known in the art. In an alternate preferred embodiment, a homology model of TPO is built, using methods known to those in the art. For example, a homology model of TPO can be made using the structure of erythropoietin (EPO) and/or other homologous four helix bundle cytokines and the sequence of human TPO. Homology models of TPO have been generated previously; see Song et. al., J. Comp. Aid. Molec. Design, 12: 419-424 (1998).

In a preferred embodiment, the results of matrix method calculations are used to identify which of the 9 amino acid positions within the epitope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each epitope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

For example, mutagenesis studies conducted on TPO have implicated the following residues in receptor binding: D8, R10, P42, F46, E50, W51, F129, R136, G137, K138, and R140; R10 and K138 appear to be especially important (see Pearce et. al. J. Biol. Chem. 272:20695-20602 (1997), Hoffman et. al. Biochemistry 35:14849-14861 (1996), Hou and Zhan Cytokine 10:319-330 (1998), Park et. al. J. Biol. Chem. 273:256-261 (1998), and Jaggerschmidt et. al. Biochem. J. 333: 729-734 (1998)). In addition, the following residues in TPO participate in disulfide bonds that are likely important in maintaining structural integrity: C7, C29, C85, and C151. Accordingly, in a preferred embodiment, these residues are not modified in the TPO variants. In an alternate preferred embodiment, only very conservative mutations are considered at these positions. Examples of conservative mutations include, but are not limited to, R10K and K138R.

In an alternate preferred embodiment, each residue that contributes significantly to the MHC binding affinity of an epitope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, energy or pseudo-energy terms describing peptide binding propensity are incorporated directly into the PDA® technology calculations. In this way, it is possible to select sequences that are active and less immunogenic in a single computational step.

In a preferred embodiment, PDA® technology and matrix method calculations are used to remove more than one MHC-binding epitope from a protein of interest.

Additional Modifications

Additional insertions, deletions, and substitutions may be incorporated into the variant TPO proteins of the invention in order to confer other desired properties.

In one embodiment, additional modifications are introduced to alter properties such as stability, solubility, and receptor binding affinity. Such modifications can also contribute to immunogenicity reduction. For example, since protein aggregates have been observed to be more immunogenic than soluble proteins, modifications that improve solubility may reduce immunogenicity (see for example Braun et. al. Pharm. Res. 14: 1472 (1997) and Speidel et. al. Eur. J. Immunol. 27: 2391 (1997)).

In one embodiment, the sequence of the variant TPO protein is modified in order to add or remove one or more N-linked or O-linked glycosylation sites. Addition of glycosylation sites to variant TPO polypeptides may be accomplished by the incorporation of one or more serine or threonine residues to the native sequence or variant TPO polypeptide (for O-linked glycosylation sites) or by the incorporation of a canonical N-linked glycosylation site, including but not limited to, N-X-Y, where X is any amino acid except for proline and Y is preferably threonine, serine or cysteine. Glycosylation sites may be removed by replacing one or more serine or threonine residues or by replacing one or more canonical N-linked glycosylation sites.

In another preferred embodiment, cysteines or other reactive amino acids are designed into variant TPO proteins in order to incorporate labeling sites or PEGylation sites.

In another preferred embodiment, the N- and C-termini of a variant TPO protein are joined to create a cyclized or circularly permutated TPO protein. Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol. Biol. 1995; 64(2-3): 121-43; Harris ME, Pace NR, Mol Biol Rep. 1995-96; 22(2-3): 115-23; Pan T, Uhlenbeck OC., 1993 Mar. 30; 125(2): 111-4; Nardulli AM, Shapiro DJ. 1993 Winter; 3(4): 247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5,1290-1300 (1996); all hereby incorporated by reference.

To produce a circularly permuted TPO protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. The circularly permuted TPO protein may be further PEGylated or glycosylated. In a further preferred embodiment PDA® technology may be used to further optimize the TPO variant, particularly in the regions created by circular permutation. These include the novel N- and C-termini, as well as the original termini and linker peptide.

In addition, a completely cyclic TPO may be generated, wherein the protein contains no termini. This is accomplished utilizing intein technology. Thus, peptides can be cyclized and in particular inteins may be utilized to accomplish the cyclization.

Variant TPO polypeptides of the present invention may also be modified to form chimeric molecules comprising a variant TPO polypeptide fused to another, heterologous polypeptide or amino acid sequence. In one embodiment, such a chimeric molecule comprises a fusion of a variant TPO polypeptide with a tag polypeptide which provides an epitope to which an anti-tag antibody can selectively bind. The epitope tag is generally placed at the amino-or carboxyl-terminus of the variant TPO polypeptide. The presence of such epitope-tagged forms of a variant TPO polypeptide can be detected using an antibody against the tag polypeptide. Also, provision of the epitope tag enables the variant TPO polypeptide to be readily purified by affinity purification using an anti-tag antibody or another type of affinity matrix that binds to the epitope tag. In an alternative embodiment, the chimeric molecule may comprise a fusion of a variant TPO polypeptide with an immunoglobulin or a particular region of an immunoglobulin. For a bivalent form of the chimeric molecule, such a fusion could be to the Fc region of an IgG molecule.

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6): 547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

Generating the Variants

Variant TPO nucleic acids and proteins of the invention may be produced using a number of methods known in the art.

Preparing Nucleic Acids Encoding the TPO Variants

In a preferred embodiment, nucleic acids encoding TPO variants are prepared by total gene synthesis, or by site-directed mutagenesis of a nucleic acid encoding wild type or variant TPO protein. Methods including template-directed ligation, recursive PCR, cassette mutagenesis, site-directed mutagenesis or other techniques that are well known in the art may be utilized.

Expression Vectors

In a preferred embodiment, an expression vector that comprises the components described below and a gene encoding a variant TPO protein is prepared. Numerous types of appropriate expression vectors and suitable regulatory sequences are known in the art for a variety of host cells. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

A preferred expression vector system is a retroviral vector system such as is generally described in PCT/US97/01019 and PCT/US97/01048, both of which are hereby expressly incorporated by reference.

Labels and Fusion Constructs

The expression vector may include a secretory leader sequence or signal peptide sequence that provides for secretion of the variant TPO protein from the host cell. Suitable secretory leader sequences that lead to the secretion of a protein are known in the art. The signal sequence typically encodes a signal peptide comprised of hydrophobic amino acids which direct the secretion of the protein from the cell, as is well known in the art. The protein is either secreted into the growth media or into the periplasmic space, located between the inner and outer membrane of the cell. For expression in bacteria, usually bacterial secretory leader sequences, operably linked to a variant TPO encoding nucleic acid, are preferred.

Transfection/Transformation

The variant TPO nucleic acids are introduced into the cells either alone or in combination with an expression vector in a manner suitable for subsequent expression of the nucleic acid. The method of introduction is largely dictated by the targeted cell type, discussed below. Exemplary methods include CaPO₄ precipitation, liposome fusion, lipofectin®, electroporation, viral infection, dextran-mediated transfection, polybrene mediated transfection, protoplast fusion, direct microinjection, etc. The variant TPO nucleic acids may stably integrate into the genome of the host cell or may exist either transiently or stably in the cytoplasm. As outlined herein, a particularly preferred method utilizes retroviral infection, as outlined in PCT US97/01019, incorporated by reference.

Cell Lines For Expressing TPO Variants

Appropriate host cells for the expression of TPO variants include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc (see the ATCC cell line catalog, hereby expressly incorporated by reference), as well as primary cell lines. In one embodiment, the cells may be additionally genetically engineered, that is, contain exogenous nucleic acid other than the expression vector comprising the variant TPO nucleic acid.

Expression Methods

The variant TPO proteins of the present invention are produced by culturing a host cell transformed with an expression vector containing nucleic acid encoding a variant TPO protein, under the appropriate conditions to induce or cause expression of the variant TPO protein. The conditions appropriate for variant TPO protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

In a preferred embodiment, TPO variants are expressed in E. coli and refolded from inclusion bodies (see Hou, J. and Zhan, H., Cytokine, 10:319-30 (1998)). Bacterial expression systems and methods for their use are well known in the art (see Current Protocols in Molecular Biology, Wiley & Sons, and Molecular Cloning—A Laboratory Manual —3rd Ed., Cold Spring Harbor Laboratory Press, New York (2001)). The choice of codons, suitable expression vectors and suitable host cells will vary depending on a number of factors, and may be easily optimized as needed.

In an alternate preferred embodiment, TPO variants are expressed in mammalian cells (see for example Kaszubska et. al., Protein Expression and Purification, 18: 213-220 (2000)) or in other expression systems including but not limited to yeast, baculovirus, and in vitro expression systems.

In one embodiment, the variant TPO nucleic acids, proteins and antibodies of the invention are labeled with a label other than the scaffold.

By “labeled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

Purification

In a preferred embodiment, the TPO variants are purified or isolated after expression. Variant TPO proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, a TPO variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3rd ed (1994). The degree of purification necessary will vary depending on the desired use, and in some instances no purification will be necessary.

Posttranslational Modification and Derivatization

Once made, the variant TPO proteins may be covalently modified. Covalent and non-covalent modifications of the protein are thus included within the scope of the present invention. Such modifications may be introduced into a variant TPO polypeptide by reacting targeted amino acid residues of the polypeptide with an organic derivatizing agent that is capable of reacting with selected side chains or terminal residues. Optimal sites for modification can be chosen using a variety of criteria, including but not limited to, visual inspection, structural analysis, sequence analysis, and molecular simulation.

In one embodiment, the variant TPO proteins of the invention are labeled with at least one element, isotope or chemical compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position. Labels include but are not limited to biotin, tag (e.g. FLAG, Myc) and fluorescent labels (e.g. fluorescein).

One type of covalent modification includes reacting targeted amino acid residues of a variant TPO polypeptide with an organic derivatizing agent that is capable of reacting with selected side chains or the N-or C-terminal residues of a variant TPO polypeptide. Derivatization with bifunctional agents is useful, for instance, for cross linking a variant TPO protein to a water-insoluble support matrix or surface for use in the method for purifying anti-variant TPO antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio] propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Such derivatization may improve the solubility, absorption, permeability across the blood brain barrier, serum half life, and the like. Modifications of variant TPO polypeptides may alternatively eliminate or attenuate any possible undesirable side effect of the protein. Moieties capable of mediating such effects are disclosed, for example, in Remington's Pharmaceutical Sciences, 16th ed., Mack Publishing Co., Easton, Pa. (1980).

Another type of covalent modification of variant TPO comprises linking the variant TPO polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337. A variety of coupling chemistries may be used to achieve PEG attachment, as is well known in the art. Examples, include but are not limited to, the technologies of Shearwater and Enzon, which allow modification at primary amines, including but not limited to, lysine groups and the N-terminus. See, Kinstler et al, Advanced Drug Deliveries Reviews, 54, 477-485 (2002) and M J Roberts et al, Advanced Drug Delivery Reviews, 54, 459-476 (2002), both hereby incorporated by reference.

Assaying the Activity of the Variants

Variant sequences that are designed to maintain thrombopoietic activity and to have reduced or eliminated immunogenicity may be tested experimentally for activity. As is known to those in the art, several methods may be used to characterize thrombopoiesis activity, including but not limited to those described below.

In a preferred embodiment, wild type and variant proteins will be analyzed for their ability to induce luciferase expression in an engineered TPO-responsive cell line, BAF-3 (see Duffy et. al., J. Med. Chem. 44:3730-3745 (2001)). Briefly, the cells are transfected with genes encoding the TPO receptor and a luciferase reporter construct. The cells are treated with varying concentrations of wild type or variant TPO, and luminescence is measured.

In a preferred embodiment, wild type and variant proteins will be analyzed for their ability to sustain viability and growth of the TPO-responsive cell line M-07e (Brizzi et. al., Br. J. Haematol., 76: 203-209 (1990)). When stimulated with TPO, the growth of this megakaryocytoma-derived cell line, which constitutively expresses c-Mpl (the TPO-receptor) and other megakaryocyte markers, can be sustained in a concentration-dependent and saturable manner. A reliable, non-radioactive indicator for cell growth is Alamar Blue, a water-soluble non-toxic fluorometric/colorimetric proliferation indicator that measures cell metabolism (Shahan et. al., J. Immunol. Meth., 175: 181-7 (1994)). Cellular growth and metabolism reduces Alamar Blue, resulting in a blue-to-red color change. Non-viable or quiescent cells do not reduce Alamar Blue and thus no color change is observed.

Determining the Immunogenicity of the Variants

In a preferred embodiment, the immunogenicity of the TPO variants is determined experimentally to confirm that the variants do have reduced or eliminated immunogenicity relative to the wild type protein.

In a preferred embodiment, ex vivo T cell activation assays are used to experimentally quantitate immunogenicity. In this method, antigen presenting cells and naïve T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et. al. J. Immunol. Meth., 24: 17-24 (2000)).

In an alternate embodiment, the TPO variants are analyzed to determine the affinity of one or more peptide regions for one or more MHC alleles. Affinity measurements can be performed in any of a number of ways, such as by using Biacore® or AlphaScreen™ technologies.

In an alternate preferred embodiment, immunogenicity is measured in transgenic mouse systems. For example, mice expressing fully or partially human Class II MHC molecules may be used. (See Andersson, E. C., Hansen, B. E., Jacobsen, H., Madsen, L. S., Andersen, C. B., Engberg, J., Rothbard, J. B., McDevitt, G. S., Malmstrom, V., Holmdahl, R., Svejgaard, A., And Fugger, L. “Definition of MHC and T cell receptor contacts in the HLA-DR4-restricted immunodominant epitope in type 11 collagen and characterization of collagen-induced arthritis in HLA-DR4 and human CD4 transgenic mice” Proc. Natl. Acad. Sci. USA, 95, 7574-7579 (1998); Taneja, V. and Chella, S. D. “Association of MHC and rheumatoid arthritis Regulatory role of HLA class 11 molecules in animal models of RA: studies on transgenic/knockout mice” Arthritis Res, 2, 205-207 (2000); Forsthuber, T. G., Shive, C. L., Wienhold, W., de Graaf, K., Spack, E. G., Sublett, R., Melms, A., Kort, J., Racke, M. K. and Weissert, R. “T Cell Epitopes of Human Myelin Oligodendrocyte Glycoprotein Identified in HLA-DR4 (DRB1*0401) Transgenic Mice Are Encephalitogenic and Are Presented by Human B Cells”, J. Immunology, 167, 7119-7125 (2001); and Paisansinsup, T., Deshmukh, U.S., Chowdhary, V. S., Luthra, H. S., Fu, S. M. and David, C. S. “HLA Class II Influences the Immune Response and Antibody Diversification to Ro60/Sjogren's Syndrome-A: Heightened Antibody Responses and Epitope Spreading in Mice Expressing HLA-DR molecules” Journal of Immunology, 168, 5876-5884 (2002).) All references cited herein are expressly incorporated in their entirety.

In an alternate embodiment, immunogenicity is tested by administering the TPO variants to one or more animals, including rodents and primates, and monitoring for antibody formation.

Administration and Treatment Using TPO Variants

Once made, the variant TPO proteins and nucleic acids of the invention find use in a number of applications. In a preferred embodiment, the variant TPO proteins are administered to a patient to treat a TPO-related disorder.

The administration of the variant TPO proteins of the present invention, preferably in the form of a sterile aqueous solution, may be done in a variety of ways, including, but not limited to, orally, parenterally, subcutaneously, intravenously, intranasally, transdermally, intraperitoneally, intramuscularly, intrapulmonary, vaginally, rectally, intranasally or intraocularly. In some instances, the variant TPO protein may be directly applied as a solution or spray. Depending upon the manner of introduction, the pharmaceutical composition may be formulated in a variety of ways. The concentration of the therapeutically active variant TPO protein in the formulation may vary from about 0.1 to 100 weight %. In another preferred embodiment, the concentration of the variant TPO protein is in the range of 0.003 to 1.0 molar, with dosages from 0.03, 0.05, 0.1, 0.2, and 0.3 millimoles per kilogram of body weight being preferred.

The pharmaceutical compositions of the present invention comprise a variant TPO protein in a form suitable for administration to a patient. In a preferred embodiment, the pharmaceutical compositions are in a water-soluble form, such as being present as pharmaceutically acceptable salts, which is meant to include both acid and base addition salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant TPO proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

In a preferred embodiment, the nucleic acid encoding the variant TPO proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where they act as inhibitors, despite their low intracellular concentrations caused by their restricted uptake by the cell membrane. [Zamecnik et al., Proc. Natl. Acad. Sci. U.S.A. 83:4143-4146 (1986)]. The oligonucleotides can be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection [Dzau et al., Trends in Biotechnology 11:205-210 (1993)]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

All references cited herein, including patents, patent applications (provisional, utility and PCT), and incorporated by reference in their entirety.

### EXAMPLES

### Example 1

### Identification of MHC-binding Epitopes in Native TPO

In order to find MHC-binding epitopes, each 9-residue fragment of native human TPO (SEQ ID NO:2) was analyzed for its propensity to bind to each of 52 Class II MHC alleles for which peptide binding affinity matrices have been derived (Sturniolo, supra). The calculations were performed using cutoffs of 1%, 3%, and 5%. The number of alleles that each peptide is predicted to bind at each of these cutoffs are shown below. 9-mer peptides that are not listed below are not predicted to bind to any alleles at the 5%, 3%, or 1% cutoffs.

| First | Last | 9-mer sequence | 1% | 3% | 5% |
|---|---|---|---|---|---|
| residue | residue | from SEQ ID NO: 2 | Hits | Hits | Hits |
| 9 | 17 | LRVLSKLLR | 17 | 31 | 36 |
| 11 | 19 | VLSKLLRDS | 9 | 14 | 17 |
| 15 | 23 | LLRDSHVLH | 5 | 6 | 7 |
| 16 | 24 | LRDSHVLHS | 4 | 13 | 21 |
| 22 | 30 | LHSRLSQCP | 0 | 0 | 1 |
| 32 | 40 | VHPLPTPVL | 0 | 0 | 1 |
| 39 | 47 | VLLPAVDFS | 0 | 0 | 4 |
| 63 | 71 | ILGAVTLLL | 0 | 3 | 9 |
| 64 | 72 | LGAVTLLLE | 0 | 0 | 1 |
| 69 | 77 | LLLEGVMAA | 2 | 8 | 14 |
| 90 | 98 | LGQLSGQVR | 0 | 0 | 2 |
| 97 | 105 | VRLLLGALQ | 6 | 25 | 32 |
| 101 | 109 | LGALQSLLG | 0 | 0 | 1 |
| 104 | 112 | LQSLLGTQL | 1 | 2 | 2 |
| 127 | 135 | IFLSFQHLL | 0 | 2 | 2 |
| 128 | 136 | FLSFQHLLR | 0 | 3 | 6 |
| 131 | 139 | FQHLLRGKV | 0 | 3 | 6 |
| 134 | 142 | LLRGKVRFL | 0 | 0 | 1 |
| 135 | 143 | LRGKVRFLM | 17 | 18 | 21 |
| 139 | 147 | VRFLMLVGG | 0 | 5 | 21 |
| 141 | 149 | FLMLVGGST | 0 | 1 | 4 |
| 142 | 150 | LMLVGGSTL | 0 | 1 | 6 |
| 144 | 152 | LVGGSTLCV | 0 | 8 | 11 |
| 152 | 160 | VRRAPPTTA | 1 | 10 | 17 |
| 167 | 175 | LVLTLNELP | 0 | 3 | 3 |
| 171 | 179 | LNELPNRTS | 0 | 0 | 1 |
| 200 | 208 | WQQGFRAKI | 0 | 0 | 2 |
| 204 | 212 | FRAKIPGLL | 2 | 3 | 6 |
| 208 | 216 | IPGLLNQTS | 0 | 0 | 2 |
| 211 | 219 | LLNQTSRSL | 0 | 0 | 6 |
| 232 | 240 | LLNGTRGLF | 0 | 1 | 2 |
| 283 | 291 | YTLFPLPPT | 0 | 1 | 1 |
| 296 | 304 | VVQLHPLLP | 3 | 8 | 12 |
| 297 | 305 | VQLHPLLPD | 1 | 5 | 10 |
| 318 | 326 | LNTSYTHSQ | 0 | 2 | 7 |
| 322 | 330 | YTHSQNLSQ | 0 | 2 | 2 |

Based on the above analysis, the 9-mer residues that are predicted to bind to the most MHC alleles are residues 9-17, 11-19, 16-24, 69-77, 97-105, 135-143, 139-147, 144-152, 152-160, 296-304, and 297-305.

Each 9-residue fragment of native human TPO (SEQ ID NO:2) was also analyzed to determine the percent of the United States population with at least one allele that binds the 9-mer peptide. The calculations were performed using a 5% cutoff.

| Start | End | Sequence from SEQ ID NO:2 | % pop |
|---|---|---|---|
| 9 | 17 | LRVLSKLLR | 58.69% |
| 11 | 19 | VLSKLLRDS | 21.21% |
| 15 | 23 | LLRDSHVLH | 21.29% |
| 16 | 24 | LRDSHVLHS | 44.64% |
| 22 | 30 | LHSRLSQCP | 1.73% |
| 32 | 40 | VHPLPTPVL | 4.96% |
| 63 | 71 | ILGAVTLLL | 33.54% |
| 69 | 77 | LLLEGVMAA | 22.70% |
| 90 | 98 | LGQLSGQVR | 0.00% |
| 97 | 105 | VRLLLGALQ | 39.93% |
| 104 | 112 | LQSLLGTQL | 16.61% |
| 127 | 135 | IFLSFQHLL | 24.75% |
| 128 | 136 | FLSFQHLLR | 20.92% |
| 131 | 139 | FQHLLRGKV | 13.23% |
| 134 | 142 | LLRGKVRFL | 1.73% |
| 135 | 143 | LRGKVRFLM | 53.69% |
| 139 | 147 | VRFLMLVGG | 49.72% |
| 141 | 149 | FLMLVGGST | 14.02% |
| 142 | 150 | LMLVGGSTL | 37.25% |
| 144 | 152 | LVGGSTLCV | 41.37% |
| 152 | 160 | VRRAPPTTA | 25.09% |
| 167 | 175 | LVLTLNELP | 13.99% |
| 171 | 179 | LNELPNRTS | 1.73% |
| 204 | 212 | FRAKIPGLL | 5.14% |
| 208 | 216 | IPGLLNQTS | 5.94% |
| 211 | 219 | LLNQTSRSL | 16.45% |
| 232 | 240 | LLNGTRGLF | 21.29% |
| 283 | 291 | YTLFPLPPT | 2.01% |
| 296 | 304 | VVQLHPLLP | 36.88% |
| 297 | 305 | VQLHPLLPD | 19.82% |
| 318 | 326 | LNTSYTHSQ | 19.10% |
| 322 | 330 | YTHSQNLSQ | 13.99% |

Based on the above analysis, the 9-mer residues that are predicted to bind to alleles that are present at least 20% of the United States population are residues 9-17, 11-19, 15-23, 16-24, 63-71, 69-77, 97-105, 127-135, 128-136, 135-143, 139-147, 142-150, 144-152, 152-160, 232-240, and 296-304.

The sequence of wild type human TPO (SEQ ID NO:2) was also compared to peptides that are known to bind human class II MHC alleles. Regions of TPO that are similar to known binders may bind to MHC molecules. The program RANKPEP (mifoundation.org/Tools/rankpep) was used to identify epitopes that may bind to the following human Class II MHC alleles: DRB1*0101, DRB1*0301, DRB1*0401, DRB1*0701, DRB1*1101, DRB1*1301, DRB1*1501, DRB4*0101, DRB5*0101, DQA1*0101/DQB1*0501, DQA1*0501/DQB1*0201, DQA1*0102/DQB1*0602, and DPA1*0201/DPB1*0901.9-mer peptides that are similar to known MHC binders include:

| START POS. | SEQUENCE FROM SEQ ID NO:2 | SCORE | % OPT. |
|---|---|---|---|
| 3 | APPACDLRV | 12 | 23.54% |
| 8 | DLRVLSKLL | 76 | 60.80% |
| 25 | RLSQCPEVH | 77 | 61.60% |
| 44 | VDFSLGEWK | 63 | 48.46% |
| 52 | KTQMEETKA | 59 | 47.20% |
| 54 | QMEETKAQD | 63 | 50.40% |
| 63 | ILGAVTLLL | 14 | 32.06% |
| 86 | LSSLLGQLS | 69 | 51.88% |
| 101 | LGALQSLLG | 61 | 45.86% |
| 104 | LQSLLGTQL | 67 | 50.38% |
| 127 | IFLSFQHLL | 9 | 21.34% |
| 128 | FLSFQHLLR | 10 | 22.62% |
| 135 | LRGKVRFLM | 10 | 14.68% |
| 139 | VRFLMLVGG | 70 | 53.85% |
| 141 | FLMLVGGST | 61 | 45.86% |
| 152 | VRRAPPTTA | 71 | 54.62% |
| 160 | AVPSRTSLV | 15 | 29.20% |
| 184 | TNFTASART | 59 | 45.38% |
| 186 | FTASARTTG | 9 | 21.32% |
| 198 | LKWQQGFRA | 18 | 27.76% |
| 199 | KWQQGFRAK | 18 | 27.37% |
| 200 | WQQGFRAKI | 11 | 16.46% |
| 215 | TSRSLDQIP | 65 | 52.00% |
| 229 | IHELLNGTR | 61 | 46.92% |
| 322 | YTHSQNLSQ | 62 | 46.62% |

These identify the region from residues 135-149 as being especially likely to contain MHC-binding epitopes.

### Example 2

### Identification of Less Immunogenic Variants of Epitopes 1-4

Several methods were used to generate alternate sequences for epitopes 1-4 that are predicted to confer decreased immunogenicity.

Altering the Three Residues that Contribute Most to MHC Binding

Here, the matrix method was used to identify which of the 9 amino acid positions within the epitope(s) contribute most to the overall binding propensities for each particular allele “hit”. This analysis considers which positions (P1-P9) are occupied by amino acids with propensity scores that are consistently large and positive for alleles scoring above the threshold values. The matrix method was then used to identify amino acid substitutions at said positions that would decrease or eliminate predicted immunogenicity. PDA® technology was used to determine which of the alternate sequences with reduced or eliminated immunogenicity are compatible with maintaining the structure and function of the protein.

Using the above approach, the following positions in the 9-17 epitope were found to make the greatest overall contribution to binding propensity scores: L9, R10, and K14. The binding score for many different alleles, and hence immunogenicity, can be decreased by incorporating mutations including, but not limited to, the following: L9A, L9C, L9D, L9E, L9G, L9H, L9K, L9N, L9P, L9Q, L9R, L9S, L9T, R10A, R1OC, R1OD, R10E, R10F, R10G, R10H, R10I, R10K, R10L, R10M, R10N, R10P, R10Q, R10S, R10T, R10W, R10Y, K14A, K14D, K14E, and K14Q. Point mutations that are especially effective in reducing immunogenicity include, but are not limited to, L9A, L9C, L9D, L9E, L9G, L9H, L9K, L9N, L9P, L9Q, L9R, L9S, L9T, R10A, R10C, R10D, and R10P. It is also possible to identify sequences that contain two or more mutations that each contributes to immunogenicity reduction.

Alternate sequences with decreased immunogenicity include, but are not limited to, those shown below. The number of hits for the 9-179mer at 1%, 3%, and 5% thresholds is shown. The number of hits for all overlapping 9mers (that is, 1-9,2-10, 3-11, 4-12, 5-13, 6-14, 7-15, 8-16, 10-18, 11-19, 12-20, 13-21, 14-22, 15-23, 16-24, and 17-25) at 1%, 3%, and 5% thresholds is also shown. The wild-type sequence and matrix scores are shown in the top row of data for reference.

| sequence | anchor | anchor | anchor | overlap | overlap | overlap |
|---|---|---|---|---|---|---|
| at residues 9-17 | 1% | 3% | 5% | 1% | 3% | 5% |
| LRVLSKLLR (SEQ ID NO: 2) | 17 | 31 | 36 | 18 | 33 | 45 |
| SRVLSKLLR (SEQ ID NO: 4) | 0 | 0 | 0 | 18 | 33 | 45 |
| KRVLSKLLR (SEQ ID NO: 5) | 0 | 0 | 0 | 18 | 33 | 45 |
| RRVLSKLLR (SEQ ID NO: 6) | 0 | 0 | 0 | 18 | 33 | 45 |
| ERVLSKLLR (SEQ ID NO: 7) | 0 | 0 | 0 | 18 | 33 | 45 |
| LDVLSKLLR (SEQ ID NO: 8) | 0 | 0 | 0 | 18 | 33 | 45 |
| LEVLSKLLR (SEQ ID NO: 9) | 0 | 6 | 9 | 18 | 33 | 45 |
| LSVLSKLLR (SEQ ID NO: 10) | 0 | 5 | 6 | 18 | 33 | 45 |
| LTVLSKLLR (SEQ ID NO: 11) | 0 | 5 | 9 | 18 | 33 | 45 |
| LRVLSELLR (SEQ ID NO: 12) | 0 | 4 | 7 | 9 | 19 | 28 |
| LRVLSDLLR (SEQ ID NO: 13) | 0 | 2 | 4 | 9 | 25 | 35 |
| LDVLSDLLR (SEQ ID NO: 14) | 0 | 0 | 0 | 9 | 25 | 35 |
| LDVLSELLR (SEQ ID NO: 15) | 0 | 0 | 0 | 9 | 19 | 28 |
| LDVLSRLLR (SEQ ID NO: 16) | 0 | 0 | 0 | 10 | 31 | 45 |
| LEVLSDLLR (SEQ ID NO: 17) | 0 | 0 | 0 | 9 | 25 | 35 |
| LEVLSELLR (SEQ ID NO: 18) | 0 | 0 | 0 | 9 | 19 | 28 |
| LEVLSRLLR (SEQ ID NO: 19) | 0 | 5 | 6 | 10 | 31 | 45 |
| LSVLSDLLR (SEQ ID NO: 20) | 0 | 0 | 0 | 9 | 25 | 35 |
| LSVLSELLR (SEQ ID NO: 21) | 0 | 0 | 0 | 9 | 19 | 28 |
| LSVLSRLLR (SEQ ID NO: 22) | 0 | 2 | 5 | 10 | 31 | 45 |
| LTVLSDLLR (SEQ ID NO: 23) | 0 | 0 | 0 | 9 | 25 | 35 |
| LTVLSELLR (SEQ ID NO: 24) | 0 | 0 | 0 | 9 | 19 | 28 |
| LTVLSRLLR (SEQ ID NO: 25) | 0 | 5 | 6 | 10 | 31 | 45 |

Using the above approach, the following positions in the 135-143 epitope make the greatest overall contribution to binding propensity scores: R136, K138, and R140. The binding score for many different alleles, and hence immunogenicity, can be decreased by incorporating mutations including, but not limited to, the following: R136A, R136C, R136D, R136E, R136F, R136G, R136H, R136I, R136K, R136L, R136M, R136N, R136P, R136Q, R136S, R136T, R136W, R136Y, K138A, K138P, R140A, R140D, R140E, and R140Q. It is also possible to identify sequences that contain two or more mutations that each contributes to immunogenicity reduction.

Alternate sequences with decreased immunogenicity include, but are not limited to, those shown below. The number of hits for the 135-439mer at 1%, 3%, and 5% thresholds is shown. The number of hits for all overlapping 9mers (that is, 127-135, 128-136, 129-137, 130-138, 131-139, 132-140, 133-141, 134-142, 136-144, 137-145,138-146, 139-147, 140-148, 141-149, 142-150, and 143-151) at 1%, 3%, and 5% thresholds is also shown. The wild-type sequence and ImmunoFilter scores are shown in the top row of data for reference.

| sequence | anchor | anchor | anchor | overlap | overlap | overlap |
|---|---|---|---|---|---|---|
| at residues 135-143 | 1% | 3% | 5% | 1% | 3% | 5% |
| LRGKVRFLM (SEQ ID NO: 2) | 17 | 18 | 21 | 0 | 15 | 46 |
| LDGKVRFLM (SEQ ID NO: 26) | 0 | 0 | 0 | 0 | 11 | 35 |
| LEGKVRFLM (SEQ ID NO: 27) | 0 | 3 | 11 | 1 | 11 | 36 |
| LQGKVRFLM (SEQ ID NO: 28) | 7 | 17 | 17 | 2 | 15 | 47 |
| LKGKVRFLM (SEQ ID NO: 29) | 6 | 16 | 17 | 1 | 14 | 46 |
| LRGKVDFLM (SEQ ID NO: 30) | 0 | 0 | 0 | 0 | 10 | 24 |
| LRGKVEFLM (SEQ ID NO: 31) | 0 | 3 | 4 | 0 | 10 | 28 |
| LRGNVDFLM (SEQ ID NO: 32) | 0 | 0 | 0 | 0 | 10 | 24 |
| LRGQVDFLM (SEQ ID NO: 33) | 0 | 0 | 0 | 0 | 10 | 24 |
| LRGSVDFLM (SEQ ID NO: 34) | 0 | 0 | 0 | 0 | 10 | 24 |
| LRGTVDFLM (SEQ ID NO: 35) | 0 | 0 | 0 | 0 | 10 | 24 |
| LRGRVDFLM (SEQ ID NO: 36) | 0 | 0 | 1 | 0 | 10 | 24 |
| LRGNVEFLM (SEQ ID NO: 37) | 0 | 0 | 0 | 0 | 10 | 28 |
| LRGSVEFLM (SEQ ID NO: 38) | 0 | 0 | 0 | 0 | 10 | 28 |
| LRGRVEFLM (SEQ ID NO: 39) | 0 | 0 | 1 | 0 | 10 | 28 |
| LRGQVEFLM (SEQ ID NO: 40) | 0 | 0 | 3 | 0 | 10 | 28 |
| LRGTVEFLM (SEQ ID NO: 41) | 0 | 0 | 0 | 0 | 10 | 28 |

Ensure Compatibility with Structure and Function

Alternate methods can also be used to identify less immunogenic sequences. Here, positions P1-P4, P6, P7, and P9 in each MHC binding epitope were analyzed to identify a subset of amino acid substitutions that are potentially compatible with maintaining the structure and function of the protein. The subset of amino acids was initially selected by visual inspection and analysis of prior mutagenesis data, discussed above.

All possible combinations of selected amino acids were then analyzed using matrix method calculations, and sequences with significantly decreased immunogenicity were identified.

Sequences that reduce or eliminate the predicted MHC binding of residues 9-17 and do not vary the functionally important residue R10 include, but are not limited to, those shown below. These sequences eliminate all hits in the 9-17 epitope and also eliminate all or nearly all of the hits in the overlapping epitopes. The wild-type sequence and matrix method scores are shown in the top row of data for reference. In all of the variants shown below, it is possible to replace A9 with alternate non-hydrophobic residues, including D, E, G, H, K, N, Q, R, S, and T.

| sequence | anchor | anchor | anchor | overlap | overlap | overlap |
|---|---|---|---|---|---|---|
| at residues 9-17 | 1% | 3% | 5% | 1% | 3% | 5% |
| LRVLSKLLR (SEQ ID NO: 2) | 17 | 31 | 36 | 18 | 33 | 45 |
| ARALSKLLE (SEQ ID NO: 42) | 0 | 0 | 0 | 0 | 0 | 0 |
| ARALSKALE (SEQ ID NO: 43) | 0 | 0 | 0 | 0 | 0 | 0 |
| ARALSKALS (SEQ ID NO: 44) | 0 | 0 | 0 | 0 | 0 | 0 |
| ARALSKALA (SEQ ID NO: 45) | 0 | 0 | 0 | 0 | 0 | 0 |
| ARALSKILE (SEQ ID NO: 46) | 0 | 0 | 0 | 0 | 0 | 0 |
| ARALSKVLE (SEQ ID NO: 47) | 0 | 0 | 0 | 0 | 0 | 0 |
| ARALSRLLE (SEQ ID NO: 48) | 0 | 0 | 0 | 0 | 0 | 0 |
| ARALSRALE (SEQ ID NO: 49) | 0 | 0 | 0 | 0 | 0 | 0 |
| ARALSRALS (SEQ ID NO: 50) | 0 | 0 | 0 | 0 | 0 | 0 |
| ARALSRALA (SEQ ID NO: 51) | 0 | 0 | 0 | 0 | 0 | 0 |
| ARALSRILE (SEQ ID NO: 52) | 0 | 0 | 0 | 0 | 0 | 0 |
| ARALSRVLE (SEQ ID NO: 53) | 0 | 0 | 0 | 0 | 0 | 0 |
| ARVLSKLLE (SEQ ID NO: 54) | 0 | 0 | 0 | 0 | 0 | 1 |
| ARVLSKALE (SEQ ID NO: 55) | 0 | 0 | 0 | 0 | 0 | 1 |
| ARVLSKILE (SEQ ID NO: 56) | 0 | 0 | 0 | 0 | 0 | 1 |
| ARVLSKVLE (SEQ ID NO: 57) | 0 | 0 | 0 | 0 | 0 | 1 |
| ARVLSRLLE (SEQ ID NO: 58) | 0 | 0 | 0 | 0 | 0 | 1 |
| ARVLSRALE (SEQ ID NO: 59) | 0 | 0 | 0 | 0 | 0 | 1 |
| ARVLSRILE (SEQ ID NO: 60) | 0 | 0 | 0 | 0 | 0 | 1 |
| ARVLSRVLE (SEQ ID NO: 61) | 0 | 0 | 0 | 0 | 0 | 1 |
| ARILSKLLE (SEQ ID NO: 62) | 0 | 0 | 0 | 0 | 0 | 1 |
| ARILSKALE (SEQ ID NO: 63) | 0 | 0 | 0 | 0 | 0 | 1 |
| ARILSKILE (SEQ ID NO: 64) | 0 | 0 | 0 | 0 | 0 | 1 |
| ARILSKVLE (SEQ ID NO: 65) | 0 | 0 | 0 | 0 | 0 | 1 |
| ARILSRLLE (SEQ ID NO: 66) | 0 | 0 | 0 | 0 | 0 | 1 |
| ARILSRALE (SEQ ID NO: 67) | 0 | 0 | 0 | 0 | 0 | 1 |
| ARILSRILE (SEQ ID NO: 68) | 0 | 0 | 0 | 0 | 0 | 1 |
| ARILSRVLE (SEQ ID NO: 69) | 0 | 0 | 0 | 0 | 0 | 1 |

It is also possible to identify sequences with reduced immunogenicity that do not include mutations at the anchor position, L9, or which include an alternate hydrophobic residue at position 9. The wild-type sequence and matrix method scores are shown in the top row of data for reference.

| sequence | anchor | anchor | anchor | overlap | overlap | overlap |
|---|---|---|---|---|---|---|
| at residues 9-17 | 1% | 3% | 5% | 1% | 3% | 5% |
| LRVLSKLLR (SEQ ID NO: 2) | 17 | 31 | 36 | 18 | 33 | 45 |
| LRALSRVLE (SEQ ID NO: 70) | 1 | 4 | 8 | 0 | 0 | 0 |
| IRALSRVLE (SEQ ID NO: 71) | 1 | 4 | 8 | 0 | 0 | 0 |
| VRALSRVLE (SEQ ID NO: 72) | 1 | 4 | 8 | 0 | 0 | 0 |
| LRALSKVLE (SEQ ID NO: 73) | 2 | 7 | 9 | 0 | 0 | 0 |
| IRALSKVLE (SEQ ID NO: 74) | 2 | 7 | 9 | 0 | 0 | 0 |
| VRALSKVLE (SEQ ID NO: 75) | 2 | 7 | 9 | 0 | 0 | 0 |
| LRALSRALE (SEQ ID NO: 76) | 4 | 6 | 14 | 0 | 0 | 0 |
| IRALSRALE (SEQ ID NO: 77) | 4 | 6 | 14 | 0 | 0 | 0 |
| VRALSRALE (SEQ ID NO: 78) | 4 | 6 | 14 | 0 | 0 | 0 |

Less immunogenic sequences were also identified for the residue 69-77 epitope. These sequences eliminate all hits in the 69-77 epitope and also eliminate nearly all of the hits in the overlapping epitopes. The wild-type sequence and matrix method scores are shown in the top row of data for reference.

| sequence | anchor | anchor | anchor | overlap | overlap | overlap |
|---|---|---|---|---|---|---|
| at residues 69-77 | 1% | 3% | 5% | 1% | 3% | 5% |
| LLLEGVMAA (SEQ ID NO: 2) | 2 | 8 | 14 | 0 | 3 | 10 |
| ALLEGVMAA (SEQ ID NO: 79) | 0 | 0 | 0 | 0 | 0 | 1 |
| ALLEGVKAA (SEQ ID NO: 80) | 0 | 0 | 0 | 0 | 0 | 1 |
| ALLEGVLAA (SEQ ID NO: 81) | 0 | 0 | 0 | 0 | 0 | 1 |
| ALLEGVQAA (SEQ ID NO: 82) | 0 | 0 | 0 | 0 | 0 | 1 |
| ALLEGAMAA (SEQ ID NO: 83) | 0 | 0 | 0 | 0 | 0 | 1 |
| ALLEGAKAA (SEQ ID NO: 84) | 0 | 0 | 0 | 0 | 0 | 1 |
| ALLEGALAA (SEQ ID NO: 85) | 0 | 0 | 0 | 0 | 0 | 1 |
| ALLEGAQAA (SEQ ID NO: 86) | 0 | 0 | 0 | 0 | 0 | 1 |
| ALLEGLMAA (SEQ ID NO: 87) | 0 | 0 | 0 | 0 | 0 | 1 |
| ALLEGLKAA (SEQ ID NO: 88) | 0 | 0 | 0 | 0 | 0 | 1 |
| ALLEGLLAA (SEQ ID NO: 89) | 0 | 0 | 0 | 0 | 0 | 1 |
| ALLEGLQAA (SEQ ID NO: 90) | 0 | 0 | 0 | 0 | 0 | 1 |
| QLLEGVMAA (SEQ ID NO: 91) | 0 | 0 | 0 | 0 | 1 | 1 |
| QLLEGVKAA (SEQ ID NO: 92) | 0 | 0 | 0 | 0 | 1 | 1 |
| QLLEGVLAA (SEQ ID NO: 93) | 0 | 0 | 0 | 0 | 1 | 1 |
| QLLEGVQAA (SEQ ID NO: 94) | 0 | 0 | 0 | 0 | 1 | 1 |
| QLLEGAMAA (SEQ ID NO: 95) | 0 | 0 | 0 | 0 | 1 | 1 |
| QLLEGAKAA (SEQ ID NO: 96) | 0 | 0 | 0 | 0 | 1 | 1 |
| QLLEGALAA (SEQ ID NO: 97) | 0 | 0 | 0 | 0 | 1 | 1 |
| QLLEGAQAA (SEQ ID NO: 98) | 0 | 0 | 0 | 0 | 1 | 1 |
| QLLEGLMAA (SEQ ID NO: 99) | 0 | 0 | 0 | 0 | 1 | 1 |
| QLLEGLKAA (SEQ ID NQ: 100) | 0 | 0 | 0 | 0 | 1 | 1 |
| QLLEGLLAA (SEQ ID NO: 101) | 0 | 0 | 0 | 0 | 1 | 1 |
| QLLEGLQAA (SEQ ID NO: 102) | 0 | 0 | 0 | 0 | 1 | 1 |
| QLLKGVMAA (SEQ ID NO: 103) | 0 | 0 | 0 | 0 | 1 | 1 |
| QLLKGVKAA (SEQ ID NO: 104) | 0 | 0 | 0 | 0 | 1 | 1 |
| QLLKGVLAA (SEQ ID NO: 105) | 0 | 0 | 0 | 0 | 1 | 1 |
| QLLKGAMAA (SEQ ID NO: 106) | 0 | 0 | 0 | 0 | 1 | 1 |
| QLLKGAKAA (SEQ ID NO: 107) | 0 | 0 | 0 | 0 | 1 | 1 |
| QLLKGALAA (SEQ ID NO: 108) | 0 | 0 | 0 | 0 | 1 | 1 |

Less immunogenic sequences were also identified for the residue 97-105 epitope. These sequences eliminate all hits in the 97-105 epitope and also eliminate nearly all of the hits in the overlapping epitopes. The wild-type sequence and matrix method scores are shown in the top row of data for reference.

| sequence | anchor | anchor | anchor | overlap | overlap | overlap |
|---|---|---|---|---|---|---|
| at residues 97-105 | 1% | 3% | 5% | 1% | 3% | 5% |
| VRLLLGALQ (SEQ ID NO: 2) | 6 | 25 | 32 | 1 | 2 | 3 |
| VKLILGALE (SEQ ID NO: 109) | 0 | 0 | 0 | 0 | 0 | 2 |
| VKVLLGALE (SEQ ID NO: 110) | 0 | 0 | 0 | 0 | 0 | 2 |
| VKVLLGSLE (SEQ ID NO: 111) | 0 | 0 | 0 | 0 | 0 | 2 |
| VKVILGALE (SEQ ID NO: 112) | 0 | 0 | 0 | 0 | 0 | 2 |
| VKVILGSLE (SEQ ID NO: 113) | 0 | 0 | 0 | 0 | 0 | 2 |
| VQVLLGALE (SEQ ID NO: 114) | 0 | 0 | 0 | 0 | 0 | 2 |
| VQVLLGSLE (SEQ ID NO: 115) | 0 | 0 | 0 | 0 | 0 | 2 |
| VQVILGALE (SEQ ID NO: 116) | 0 | 0 | 0 | 0 | 0 | 2 |
| IKLILGALE (SEQ ID NO: 117) | 0 | 0 | 0 | 0 | 0 | 2 |
| IKVLLGALE (SEQ ID NO: 118) | 0 | 0 | 0 | 0 | 0 | 2 |
| IKVLLGSLE (SEQ ID NO: 119) | 0 | 0 | 0 | 0 | 0 | 2 |
| IKVILGALE (SEQ ID NO: 120) | 0 | 0 | 0 | 0 | 0 | 2 |
| IKVILGSLE (SEQ ID NO: 121) | 0 | 0 | 0 | 0 | 0 | 2 |
| IQVLLGALE (SEQ ID NO: 122) | 0 | 0 | 0 | 0 | 0 | 2 |
| IQVLLGSLE (SEQ ID NO: 123) | 0 | 0 | 0 | 0 | 0 | 2 |
| IQVILGALE (SEQ ID NO: 124) | 0 | 0 | 0 | 0 | 0 | 2 |
| TRLLLGALE (SEQ ID NO: 125) | 0 | 0 | 0 | 0 | 0 | 2 |
| TRLLLGSLE (SEQ ID NO: 126) | 0 | 0 | 0 | 0 | 0 | 2 |
| TRLILGALE (SEQ ID NO: 127) | 0 | 0 | 0 | 0 | 0 | 2 |
| TRLILGSLE (SEQ ID NO: 128) | 0 | 0 | 0 | 0 | 0 | 2 |
| TRILLGALE (SEQ ID NO: 129) | 0 | 0 | 0 | 0 | 0 | 2 |
| TRILLGSLE (SEQ ID NO: 130) | 0 | 0 | 0 | 0 | 0 | 2 |
| TRIILGALE (SEQ ID NO: 131) | 0 | 0 | 0 | 0 | 0 | 2 |
| TRIILGSLE (SEQ ID NO: 132) | 0 | 0 | 0 | 0 | 0 | 2 |
| TRVLLGALE (SEQ ID NO: 133) | 0 | 0 | 0 | 0 | 0 | 2 |
| TRVLLGSLE (SEQ ID NO: 134) | 0 | 0 | 0 | 0 | 0 | 2 |
| TRVILGALE (SEQ ID NO: 135) | 0 | 0 | 0 | 0 | 0 | 2 |
| TRVILGSLE (SEQ ID NO: 136) | 0 | 0 | 0 | 0 | 0 | 2 |
| TKLLLGALE (SEQ ID NO: 137) | 0 | 0 | 0 | 0 | 0 | 2 |
| TKLLLGSLE (SEQ ID NO: 138) | 0 | 0 | 0 | 0 | 0 | 2 |
| TKLILGALE (SEQ ID NO: 139) | 0 | 0 | 0 | 0 | 0 | 2 |
| TKLILGSLE (SEQ ID NO: 140) | 0 | 0 | 0 | 0 | 0 | 2 |
| TKILLGALE (SEQ ID NO: 141) | 0 | 0 | 0 | 0 | 0 | 2 |
| TKILLGSLE (SEQ ID NO: 142) | 0 | 0 | 0 | 0 | 0 | 2 |
| TKIILGALE (SEQ ID NO: 143) | 0 | 0 | 0 | 0 | 0 | 2 |
| TKIILGSLE (SEQ ID NO: 144) | 0 | 0 | 0 | 0 | 0 | 2 |
| TKVLLGALE (SEQ ID NO: 145) | 0 | 0 | 0 | 0 | 0 | 2 |
| TKVLLGSLE (SEQ ID NO: 146) | 0 | 0 | 0 | 0 | 0 | 2 |
| TKVILGALE (SEQ ID NO: 147) | 0 | 0 | 0 | 0 | 0 | 2 |
| TKVILGSLE (SEQ ID NO: 148) | 0 | 0 | 0 | 0 | 0 | 2 |
| TQLLLGALE (SEQ ID NO: 149) | 0 | 0 | 0 | 0 | 0 | 2 |
| TQLLLGSLE (SEQ ID NO: 150) | 0 | 0 | 0 | 0 | 0 | 2 |
| TQLILGALE (SEQ ID NO: 151) | 0 | 0 | 0 | 0 | 0 | 2 |
| TQLILGSLE (SEQ ID NO: 152) | 0 | 0 | 0 | 0 | 0 | 2 |
| TQILLGALE (SEQ ID NO: 153) | 0 | 0 | 0 | 0 | 0 | 2 |
| TQILLGSLE (SEQ ID NO: 154) | 0 | 0 | 0 | 0 | 0 | 2 |
| TQIILGALE (SEQ ID NO: 155) | 0 | 0 | 0 | 0 | 0 | 2 |
| TQIILGSLE (SEQ ID NO: 156) | 0 | 0 | 0 | 0 | 0 | 2 |
| TQVLLGALE (SEQ ID NO: 157) | 0 | 0 | 0 | 0 | 0 | 2 |
| TQVLLGSLE (SEQ ID NO: 158) | 0 | 0 | 0 | 0 | 0 | 2 |
| TQVILGALE (SEQ ID NO: 159) | 0 | 0 | 0 | 0 | 0 | 2 |
| TQVILGSLE (SEQ ID NO: 160) | 0 | 0 | 0 | 0 | 0 | 2 |

Finally, less immunogenic sequences were identified for the residue 135-143 epitope. These sequences conserve the identity of several residues that have been implicated in TPO function: R136, K138, and R140. The wild-type sequence and matrix method scores are shown in the top row of data for reference. These sequences eliminate all hits in the 135-143 epitope and also eliminate many of the hits in the overlapping epitopes. The wild-type sequence and matrix scores are shown in the top row of data for reference.

| sequence | anchor | anchor | anchor | overlap | overlap | overlap |
|---|---|---|---|---|---|---|
| at residues 135-143 | 1% | 3% | 5% | 1% | 3% | 5% |
| LRGKVRFLM (SEQ ID NO: 2) | 17 | 18 | 21 | 0 | 15 | 46 |
| ARGKVKHLL (SEQ ID NO: 161) | 0 | 0 | 0 | 0 | 7 | 16 |
| ARGKVKLLL (SEQ ID NO: 162) | 0 | 0 | 0 | 0 | 7 | 17 |
| ARGKVKHLM (SEQ ID NO: 163) | 0 | 0 | 0 | 0 | 7 | 18 |
| ARGKVKLLM (SEQ ID NO: 164) | 0 | 0 | 0 | 0 | 7 | 19 |
| ARGKVRHLL (SEQ ID NO: 165) | 0 | 0 | 0 | 0 | 7 | 20 |
| ARGKVKFLQ (SEQ ID NO: 166) | 0 | 0 | 0 | 0 | 7 | 20 |
| ARGKVKHLQ (SEQ ID NO: 167) | 0 | 0 | 0 | 0 | 7 | 20 |
| ARGKVKLLQ (SEQ ID NO: 168) | 0 | 0 | 0 | 0 | 7 | 20 |
| ARGKVKYLQ (SEQ ID NO: 169) | 0 | 0 | 0 | 0 | 7 | 20 |
| ARGKVRHLM (SEQ ID NO: 170) | 0 | 0 | 0 | 0 | 7 | 22 |
| ARGKVRHLQ (SEQ ID NO: 171) | 0 | 0 | 0 | 0 | 7 | 24 |
| ARGKVKFLL (SEQ ID NO: 172) | 0 | 0 | 0 | 0 | 8 | 17 |
| ARGKVKYLL (SEQ ID NO: 173) | 0 | 0 | 0 | 0 | 8 | 17 |
| ARGKVKFLM (SEQ ID NO: 174) | 0 | 0 | 0 | 0 | 8 | 22 |
| ARGKVKYLM (SEQ ID NO: 175) | 0 | 0 | 0 | 0 | 8 | 22 |
| ARGKVRFLQ (SEQ ID NO: 176) | 0 | 0 | 0 | 0 | 12 | 41 |
| ARGKVRYLQ (SEQ ID NO: 177) | 0 | 0 | 0 | 0 | 12 | 41 |
| ARGKVRFLL (SEQ ID NO: 178) | 0 | 0 | 0 | 0 | 13 | 38 |
| ARGKVRYLL (SEQ ID NO: 179) | 0 | 0 | 0 | 0 | 13 | 38 |
| ARGKVRFLM (SEQ ID NO: 180) | 0 | 0 | 0 | 0 | 13 | 43 |
| ARGKVRYLM (SEQ ID NO: 181) | 0 | 0 | 0 | 0 | 13 | 43 |

It is also possible to identify sequences with reduced immunogenicity that maintain the hydrophobicity of the anchor position, L135. The wild-type sequence and matrix scores are shown in the top row of data for reference.

| sequence | anchor | anchor | anchor | overlap | overlap | overlap |
|---|---|---|---|---|---|---|
| at residues 135-143 | 1% | 3% | 5% | 1% | 3% | 5% |
| LRGKVRFLM (SEQ ID NO: 2) | 17 | 18 | 21 | 0 | 15 | 46 |
| LRGKVKYLL (SEQ ID NO: 182) | 2 | 17 | 17 | 0 | 10 | 19 |
| IRGKVKYLL (SEQ ID NO: 183) | 2 | 17 | 17 | 0 | 10 | 19 |
| VRGKVKYLL (SEQ ID NO: 184) | 2 | 17 | 17 | 0 | 12 | 22 |
| FRGKVRYLL (SEQ ID NO: 185) | 6 | 10 | 13 | 0 | 13 | 39 |
| FRGKVRHLL (SEQ ID NO: 186) | 8 | 11 | 18 | 0 | 7 | 21 |
| LRGKVKHLL (SEQ ID NO: 187) | 10 | 17 | 17 | 0 | 9 | 18 |
| IRGKVKHLL (SEQ ID NO: 188) | 10 | 17 | 17 | 0 | 9 | 18 |
| VRGKVKHLL (SEQ ID NO: 189) | 10 | 17 | 17 | 0 | 11 | 21 |
| LRGKVKFLL (SEQ ID NO: 190) | 14 | 17 | 17 | 0 | 10 | 19 |
| IRGKVKFLL (SEQ ID NO: 191) | 14 | 17 | 17 | 0 | 10 | 19 |
| VRGKVKFLL (SEQ ID NO: 192) | 14 | 17 | 17 | 0 | 12 | 22 |

| sequence | anchor | anchor | anchor | overlap | overlap | overlap |
|---|---|---|---|---|---|---|
| at residues 135-143 | 1% | 3% | 5% | 1% | 3% | 5% |
| LRGKVRFLM (SEQ ID NO: 2) | 17 | 18 | 21 | 0 | 15 | 46 |
| LRGKVRFLN (SEQ ID NO: 193) | 3 | 17 | 17 | 0 | 14 | 39 |
| LRGKVRDLM (SEQ ID NO: 194) | 0 | 6 | 14 | 0 | 9 | 21 |
| LRGKVRDLN (SEQ ID NO: 195) | 0 | 1 | 3 | 0 | 9 | 18 |
| LRGKVRDLL (SEQ ID NO: 196) | 0 | 0 | 3 | 0 | 9 | 19 |
| LRGKVRTLM (SEQ ID NO: 197) | 4 | 13 | 18 | 0 | 9 | 24 |
| LRGKVRTLN (SEQ ID NO: 198) | 0 | 4 | 5 | 0 | 9 | 21 |
| LRGKVRTLL (SEQ ID NO: 199) | 1 | 1 | 10 | 0 | 9 | 22 |
| LRGKVRQLM (SEQ ID NO: 200) | 10 | 17 | 18 | 0 | 9 | 24 |
| LRGKVRQLN (SEQ ID NO: 201) | 3 | 6 | 13 | 0 | 9 | 21 |
| LRGKVRQLL (SEQ ID NO: 202) | 1 | 12 | 15 | 0 | 9 | 22 |
| LRDKVRDLM (SEQ ID NO: 203) | 0 | 0 | 0 | 0 | 12 | 22 |
| LRDKVRDLN (SEQ ID NO: 204) | 0 | 0 | 0 | 0 | 12 | 19 |
| LRDKVRDLL (SEQ ID NO: 205) | 0 | 0 | 0 | 0 | 12 | 20 |
| LRDKVRTLM (SEQ ID NO: 206) | 0 | 1 | 1 | 0 | 12 | 25 |
| LRDKVRTLN (SEQ ID NO: 207) | 0 | 0 | 0 | 0 | 12 | 22 |
| LRDKVRTLL (SEQ ID NO: 208) | 0 | 0 | 1 | 0 | 12 | 23 |
| LRDKVRQLM (SEQ ID NO: 209) | 0 | 1 | 7 | 0 | 12 | 25 |
| LRDKVRQLN (SEQ ID NO: 210) | 0 | 1 | 2 | 0 | 12 | 22 |
| LRDKVRQLL (SEQ ID NO: 211) | 0 | 0 | 0 | 0 | 12 | 23 |

Additional sequences with reduced immunogenicity were identified that conserve L135 and retain positively charged residues at positions 136, 138, and 140.

| sequence | anchor | anchor | anchor | overlap | overlap | overlap |
|---|---|---|---|---|---|---|
| at residues 135-143 | 1% | 3% | 5% | 1% | 3% | 5% |
| LRGKVRFLM (SEQ ID NO:2) | 17 | 18 | 21 | 0 | 15 | 46 |
| LKGKVRKLL (SEQ ID NO:212) | 0 | 2 | 4 | 1 | 7 | 17 |
| LKGKVRQLL (SEQ ID NO:213) | 0 | 0 | 2 | 1 | 7 | 17 |
| LKGKVRYLL (SEQ ID NO:214) | 0 | 0 | 2 | 1 | 9 | 21 |
| LKGKVKQLL (SEQ ID NO:215) | 0 | 1 | 4 | 1 | 7 | 16 |
| LKAKVRKLL (SEQ ID NO:216) | 0 | 1 | 3 | 1 | 13 | 31 |
| LKAKVRQLL (SEQ ID NO:217) | 0 | 0 | 1 | 1 | 13 | 31 |
| LKAKVRYLL (SEQ ID NO:218) | 0 | 0 | 2 | 1 | 15 | 35 |
| LKAKVKQLL (SEQ ID NO:219) | 0 | 0 | 3 | 1 | 13 | 22 |
| LKAKVKYLL (SEQ ID NO:220) | 0 | 1 | 4 | 1 | 13 | 23 |

To obtain a greater reduction in predicted immunogenicity, mutations in residues 135-143 were combined with mutations in residues 127-134 and/or residues 144-151. The wild-type sequence and matrix method scores are shown in the top row of data for each reference.

| sequence | anchor | anchor | anchor | overlap | overlap | overlap |
|---|---|---|---|---|---|---|
| at residues 129-145 | 1% | 3% | 5% | 1% | 3% | 5% |
| LSFQHLLRGKVRFLMLV (SEQ ID NO:2) | 17 | 18 | 21 | 0 | 23 | 57 |
| ESFEHLLKGKVRQLLEA (SEQ ID NO:221) | 0 | 0 | 2 | 0 | 0 | 1 |
| ESFEHLLKGKVRYLLEA (SEQ ID NO:222) | 0 | 0 | 2 | 0 | 0 | 1 |
| ESFEHLARGKVRYLMEA (SEQ ID NO:223) | 0 | 0 | 0 | 0 | 0 | 1 |
| ESFEHLARGKVKFLMEA (SEQ ID NO:224) | 0 | 0 | 0 | 0 | 0 | 1 |

### Example 3

### Homology Modeling of TPO

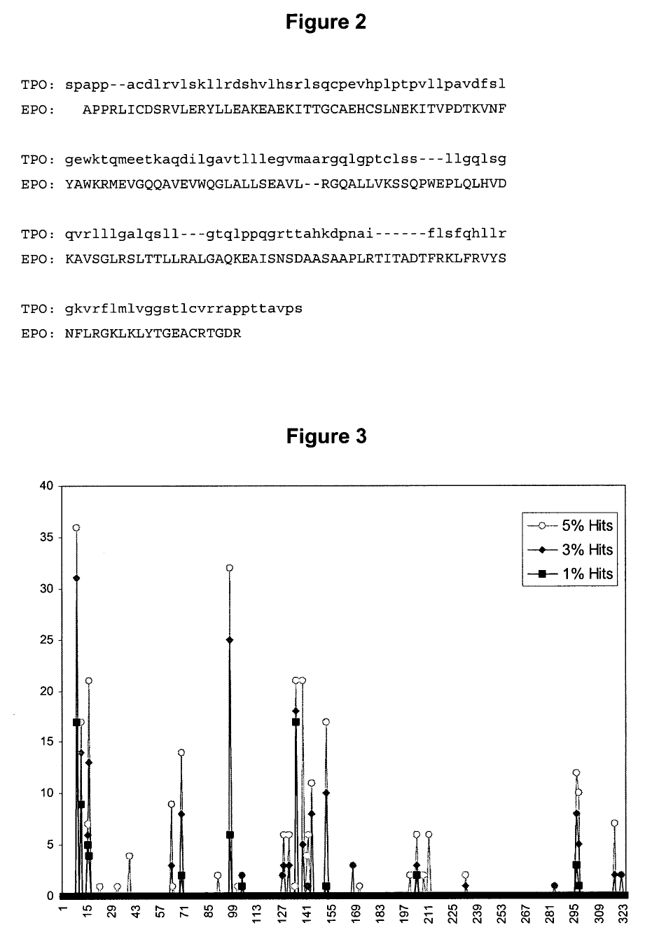

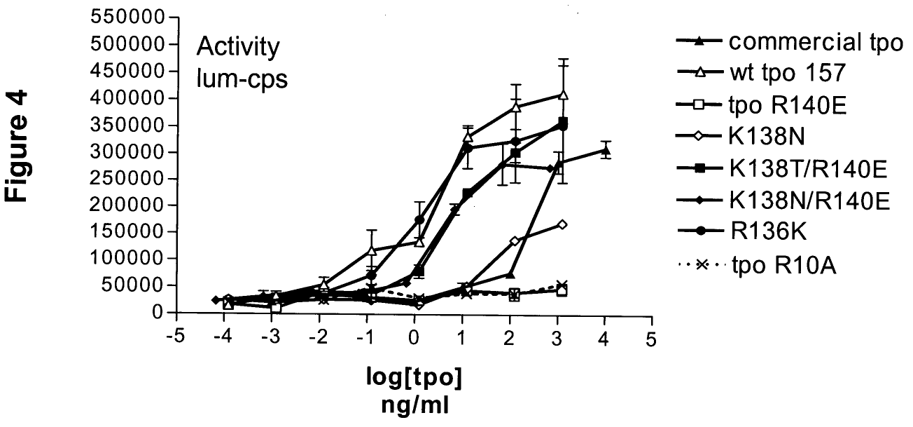

A model of the three-dimensional structure of TPO was generated using the Homology module in the computer program InsightII. The crystal structure of erythropoietin (PDB code 1 EER, Syed et. al. Nature 395:511 (1998)) and the sequence of TPO shown in FIG. 1 were used to produce the homology model. As TPO and EPO share limited sequence similarity, the correct alignment between the two sequences is somewhat ambiguous. A number of possible alignments were tested, and the sequence alignment shown in FIG. 2 was observed to produce the highest quality models.

### Example 4

### Identification of Structured, Less Immunogenic TPO Variants

PDA® calculations were performed to predict the energies of each of the less immunogenic variants of the major epitopes in TPO, as well as the native sequence. The energies of the native sequences were then compared with the energies of the variants to determine which of the less immunogenic TPO sequences are compatible with maintaining the structure and function of TPO. Each calculation used one or more of the homology models produced above as the template. Unless otherwise noted, the nine residues comprising an epitope of interest were determined to be the variable residue positions. A variety of rotameric states were considered for each variable position, and the sequence was constrained to be the sequence of a specific less immunogenic variant identified previously. Rotamer-template and rotamer-rotamer energies were then calculated using a force field including terms describing van der Waals interactions, hydrogen bonds, electrostatics, and solvation. The optimal rotameric configurations for each sequence were determined using DEE as a combinatorial optimization method.

In general, all of the sequences whose energies are similar to or better than (that is, less than) the energy of the native sequence are likely to be structured. Sequences that conserve those residues that are known to be important for function are likely to also be active. Alternatively, it is possible to model the interaction of TPO with mpl receptor and then to determine which variant sequences are compatible with forming this interaction.

Shown below is the calculated immunogenicity and energy of the native sequence and several less immunogenic variants of epitope 1 (residues 9-17). Energies were calculated using two different homology models; although the exact values vary the overall trends are fairly consistent.

| sequence | | | | | | | 5_2 | 8_2 |
|---|---|---|---|---|---|---|---|---|
| at residues 9-17 | a1% | a3% | a5% | o1% | o3% | o5% | energy | energy |
| LRVLSKLLR (SEQ ID NO:2) | 17 | 31 | 36 | 18 | 33 | 45 | 22.25 | 212.08 |
| KRVLSKLLK (SEQ ID NO:225) | 0 | 0 | 0 | 0 | 15 | 25 | 17.32 | 209.67 |
| KRVLSKLLQ (SEQ ID NO:226) | 0 | 0 | 0 | 0 | 11 | 21 | 16.86 | 206.04 |
| ARALSKALE (SEQ ID NO:43) | 0 | 0 | 0 | 0 | 0 | 0 | −12.16 | −7.53 |
| ARALSKALS (SEQ ID NO:44) | 0 | 0 | 0 | 0 | 0 | 0 | −10.62 | −7.28 |
| ARALSKVLE (SEQ ID NO:47) | 0 | 0 | 0 | 0 | 0 | 0 | −13.19 | −1.84 |
| ARALSRALS (SEQ ID NO:50) | 0 | 0 | 0 | 0 | 0 | 0 | −12.77 | −8.02 |
| ARALSRVLE (SEQ ID NO:53) | 0 | 0 | 0 | 0 | 0 | 0 | −14.98 | −3.03 |
| ARILSKALE (SEQ ID NO:63) | 0 | 0 | 0 | 0 | 0 | 1 | −13.81 | −8.47 |
| ARILSKVLE (SEQ ID NO:65) | 0 | 0 | 0 | 0 | 0 | 1 | −14.48 | −2.95 |
| ARILSRALE (SEQ ID NO:67) | 0 | 0 | 0 | 0 | 0 | 1 | −15.08 | −10.52 |
| ARILSRLLE (SEQ ID NO:66) | 0 | 0 | 0 | 0 | 0 | 1 | 20.09 | 211.32 |
| ARILSRVLE (SEQ ID NO:69) | 0 | 0 | 0 | 0 | 0 | 1 | −15.75 | −5.02 |
| ARVLSKALE (SEQ ID NO:55) | 0 | 0 | 0 | 0 | 0 | 1 | −14.41 | −8.87 |
| ARVLSKLLE (SEQ ID NO:54) | 0 | 0 | 0 | 0 | 0 | 1 | 20.82 | 212.96 |
| ARVLSKVLE (SEQ ID NO:57) | 0 | 0 | 0 | 0 | 0 | 1 | −15.11 | −3.38 |
| ARVLSRALE (SEQ ID NO:59) | 0 | 0 | 0 | 0 | 0 | 1 | −15.68 | −11.34 |
| ARVLSRVLE (SEQ ID NO:61) | 0 | 0 | 0 | 0 | 0 | 1 | −16.38 | −5.85 |

Shown below is the calculated immunogenicity and energy of the native sequence and several less immunogenic variants of epitope 2 (residues 135-143). Energies were calculated using two different homology models; although the exact values vary the overall trends are fairly consistent. In calculations for the last group of variants, residues 129, 132, and 135-145 were all treated as variable positions.

| | | | | | | 5_2 | 8_1 |
|---|---|---|---|---|---|---|---|
| a1% | a3% | a5% | o1% | o3% | o5% | energy | energy |

Sequence at residues 135-143

| | | | | | | | | |
|---|---|---|---|---|---|---|---|---|
| LRGKVRFLM (SEQ ID NO:2) | 17 | 18 | 21 | 0 | 15 | 46 | −84.72 | −88.95 |
| LKGKVRYLL (SEQ ID NO:214) | 0 | 0 | 2 | 1 | 14 | 41 | −83.52 | −87.19 |
| LKGKVRQLL (SEQ ID NO:213) | 0 | 0 | 2 | 1 | 8 | 22 | −81.62 | −85.05 |
| LKGKLRYLL (SEQ ID NO:227) | 0 | 0 | 2 | 0 | 14 | 41 | −85.41 | −79.90 |
| LKGKLRQLL (SEQ ID NO:228) | 0 | 0 | 2 | 0 | 8 | 22 | −83.66 | −77.51 |
| ARGKVRYLM (SEQ ID NO:281) | 0 | 0 | 0 | 0 | 13 | 43 | −75.61 | −79.56 |
| ARGKVKFLM (SEQ ID NO:174) | 0 | 0 | 0 | 0 | 8 | 22 | −80.59 | −81.54 |
| ARGKVKFLL (SEQ ID NO:172) | 0 | 0 | 0 | 0 | 8 | 17 | −79.54 | −79.06 |
| ARGKVKHLM (SEQ ID NO:163) | 0 | 0 | 0 | 0 | 7 | 18 | −76.79 | −79.55 |
| ARGKVKLLM (SEQ ID NO:164) | 0 | 0 | 0 | 0 | 7 | 19 | −83.70 | −82.41 |
| ARGKVKLLL (SEQ ID NO:162) | 0 | 0 | 0 | 0 | 7 | 17 | −82.65 | −79.94 |
| ARGKVKYLM (SEQ ID NO:175) | 0 | 0 | 0 | 0 | 8 | 22 | −83.26 | −83.42 |
| ARGKVKYLL (SEQ ID NO:173) | 0 | 0 | 0 | 0 | 8 | 17 | −82.21 | −80.94 |

Sequence at residues 129-145

| | | | | | | | | |
|---|---|---|---|---|---|---|---|---|
| LSFQHLLRGKVRFLMLV (SEQ ID NO:2) | 17 | 18 | 21 | 0 | 23 | 57 | −89.13 | 37.40 |
| ESFEHLLRGKVRFLMLV (SEQ ID NO:229) | 17 | 18 | 21 | 0 | 15 | 44 | −103.33 | −45.78 |
| LSFQHLLRGKVRFLMEA (SEQ ID NO:230) | 17 | 18 | 21 | 0 | 8 | 15 | −90.88 | 38.74 |
| ESFEHLLKGKVRQLLEA (SEQ ID NO:221) | 0 | 0 | 2 | 0 | 0 | 1 | −102.01 | −40.98 |
| ESFEHLLKGKVRYLLEA (SEQ ID NO:222) | 0 | 0 | 2 | 0 | 0 | 1 | −104.90 | −42.21 |
| ESFEHLARGKVRYLMEA (SEQ ID NO:223) | 0 | 0 | 0 | 0 | 0 | 1 | −95.81 | −35.14 |
| ESFEHLARGKVKFLMEA (SEQ ID NO:224) | 0 | 0 | 0 | 0 | 0 | 1 | −94.75 | −35.21 |

Shown below is the calculated immunogenicity and energy of the native sequence and several less immunogenic variants of epitope 3 (residues 69-77). Energies were calculated using two different homology models; although the exact values vary the overall trends are fairly consistent.

| sequence | | | | | | | 5_2 | 8_1 |
|---|---|---|---|---|---|---|---|---|
| at residues 69-77 | a1% | a3% | a5% | o1% | o3% | o5% | energy | energy |
| LLLEGVMAA (SEQ ID NO:2) | 2 | 8 | 14 | 0 | 3 | 10 | −56.87 | −59.30 |
| LLLEGLMAA (SEQ ID NO:231) | 0 | 0 | 2 | 0 | 3 | 10 | −52.91 | −61.31 |
| LLLEGVKAA (SEQ ID NO:232) | 0 | 2 | 3 | 0 | 3 | 10 | −55.73 | −61.60 |
| LLLEGVQAA (SEQ ID NO:233) | 0 | 2 | 3 | 0 | 3 | 10 | −57.02 | −61.18 |
| LLLEGAMAA (SEQ ID NO:234) | 0 | 2 | 4 | 0 | 3 | 10 | −49.09 | −51.72 |
| ALLEGVLAA (SEQ ID NO:81) | 0 | 0 | 0 | 0 | 0 | 1 | −55.66 | −52.58 |
| ALLEGVQAA (SEQ ID NO:82) | 0 | 0 | 0 | 0 | 0 | 1 | −54.73 | −54.20 |
| ALLEGVMAA (SEQ ID NO:79) | 0 | 0 | 0 | 0 | 0 | 1 | −54.58 | −52.54 |
| QLLEGVQAA (SEQ ID NO:94) | 0 | 0 | 0 | 0 | 1 | 1 | −54.41 | −56.74 |
| QLLEGVMAA (SEQ ID NO:91) | 0 | 0 | 0 | 0 | 1 | 1 | −54.27 | −54.95 |
| ALLEGVKAA (SEQ ID NO:80) | 0 | 0 | 0 | 0 | 0 | 1 | −53.44 | −54.77 |
| QLLEGVKAA (SEQ ID NO:92) | 0 | 0 | 0 | 0 | 1 | 1 | −53.07 | −57.17 |
| QLLKGVLAA (SEQ ID NO:105) | 0 | 0 | 0 | 0 | 1 | 1 | −52.61 | −55.71 |
| QLLKGVMAA (SEQ ID NO:103) | 0 | 0 | 0 | 0 | 1 | 1 | −52.00 | −55.55 |
| ALLEGLLAA (SEQ ID NO:89) | 0 | 0 | 0 | 0 | 0 | 1 | −51.78 | −54.66 |
| ALLEGLQAA (SEQ ID NO:90) | 0 | 0 | 0 | 0 | 0 | 1 | −50.74 | −56.24 |
| QLLKGVKAA (SEQ ID NO:104) | 0 | 0 | 0 | 0 | 1 | 1 | −50.73 | −56.14 |
| ALLEGLMAA (SEQ ID NO:87) | 0 | 0 | 0 | 0 | 0 | 1 | −50.62 | −54.56 |
| QLLEGLMAA (SEQ ID NO:99) | 0 | 0 | 0 | 0 | 1 | 1 | −50.31 | −56.96 |

Shown below is the calculated immunogenicity and energy of the native sequence and several less immunogenic variants of epitope 4 (residues 97-105). Energies were calculated using two different homology models; although the exact values vary the overall trends are fairly consistent.

| sequence | | | | | | | 5_2 | 8_1 |
|---|---|---|---|---|---|---|---|---|
| at residues 97-105 | a1% | a3% | a5% | o1% | o3% | o5% | energy | energy |
| VRLLLGALQ (SEQ ID NO:2) | 6 | 25 | 32 | 1 | 2 | 5 | −71.58 | −63.96 |
| TKILLGSLE (SEQ ID NO:142) | 0 | 0 | 0 | 0 | 0 | 4 | −66.25 | −60.24 |
| TKLLLGSLE (SEQ ID NO:138) | 0 | 0 | 0 | 0 | 0 | 4 | −65.64 | −60.07 |
| TKVLLGSLE (SEQ ID NO:146) | 0 | 0 | 0 | 0 | 0 | 4 | −66.61 | −60.03 |
| TRILLGSLE (SEQ ID NO:130) | 0 | 0 | 0 | 0 | 0 | 4 | −66.10 | −63.39 |
| TRLLLGSLE (SEQ ID NO:126) | 0 | 0 | 0 | 0 | 0 | 4 | −66.10 | −64.57 |
| TRLLLGSLQ (SEQ ID NO:235) | 0 | 0 | 0 | 1 | 2 | 5 | −68.59 | −60.87 |
| TRVLLGSLE (SEQ ID NO:134) | 0 | 0 | 0 | 0 | 0 | 4 | −67.29 | −64.65 |
| VKLILGALE (SEQ ID NO:109) | 0 | 0 | 0 | 0 | 0 | 4 | −65.45 | −64.31 |
| VKLILGALQ (SEQ ID NO:236) | 0 | 1 | 4 | 1 | 2 | 5 | −67.91 | −60.62 |
| VKVILGALE (SEQ ID NO:112) | 0 | 0 | 0 | 0 | 0 | 4 | −65.48 | −63.87 |
| VKVILGSLE (SEQ ID NO:113) | 0 | 0 | 0 | 0 | 0 | 4 | −69.69 | −63.87 |
| VKVLLGALE (SEQ ID NO:110) | 0 | 0 | 0 | 0 | 0 | 4 | −69.17 | −62.15 |
| VKVLLGSLE (SEQ ID NO:111) | 0 | 0 | 0 | 0 | 0 | 4 | −73.35 | −66.03 |
| VQVLLGALE (SEQ ID NO:114) | 0 | 0 | 0 | 0 | 0 | 2 | −67.72 | −62.42 |
| VQVLLGALQ (SEQ ID NO:237) | 0 | 1 | 4 | 1 | 2 | 3 | −70.37 | −58.84 |
| VQVLLGSLE (SEQ ID NO:115) | 0 | 0 | 0 | 0 | 0 | 2 | −71.90 | −66.30 |

### Example 5

### Activity of Reduced-Immunogenicity TPO Variants

Activity of the variant TPO molecules was determined by assaying a TPO-sensitive cell line for proliferation.

In a preferred embodiment, activity of the variant TPO molecules is determined in another cell line, such as the mouse IL-3 dependent BaF3, which has been engineered to express the human TPO receptor (See Bartley T D, Bogenberger J, Hunt P et al. Identification and cloning of a megakaryocyte growth and development factor (MGDF) that is a ligand for the cytokine receptor Mpl. Cell. 1994; 77:1117-1124; Duffy, et. al, Hydrazinonaphthalene and Azonaphthalene Thrombopoietin Mimics Are Nonpeptidyl Promoters of Megakaryocytopoiesis, J. Med. Chem. 2001, 44, 3730-3745; and de Sauvage F J, et al. Stimulation of megakaryocytopoiesis and thrombopoiesis by the c-Mpl ligand. Nature. 1994 Jun. 16;369(6481): 533-8, herein expressly incorporated by reference.). BaF3 is a mouse hematopoietic cell line, which requires IL-3 for growth and survival. When the human TPO receptor is expressed in these cells, replacement of IL-3 with TPO results in a concentration dependent response, which correlates with the specific activity of the TPO molecule.

This response can be measured with a variety of methods well known in the art. A preferred method is to measure BaF3-TPOR viability in response to TPO using Cell TiterGlo™ luminescence assay (Promega Corp. technical bulletin no. 288). Upon removal of IL-3 from the BaF3 TPO R cells for a period of 16 hours, the cells are then exposed to a range of concentrations of variant TPO for 48 hours. In a preferred method, 500 BaF3 TPOR cells are used in a volume of 25 ul per well of a 384 well assay plate. Following the 48 hour TPO exposure, viability is measured by lysing the cells in an equal volume of an enzymatic light emitting solution (luciferase) lacking only ATP to complete the reaction. Since intracellular ATP levels are directly proportional to cell number, the lysed cells provide ATP to complete the reaction. Measuring luminescence then determines TPO activity in a dose dependent and saturable fashion. The luminescence data can be analyzed several ways but a preferred embodiment is to use a variable slope non-linear regression calculation (Hill equation —REF, Prism REF). From this analysis a common measure of activity is to report the effective concentration of TPO, which yields 50% of the total activity measured (EC50). This value is therefore inversely related to the specific activity of the variant TPO and in a preferred embodiment is normalized to the wild-type EC50 value.

The activity of variant TPO proteins with mutations in residues 9-17 and 135-143 are shown in the table below. The variants were selected to modify the residues that are predicted to contribute most to MHC-binding affinity.

| TPO variant | EC50 |
|---|---|
| wt TPO | 0.069 |
| R136K | 0.092 |
| K138T/R140E | 0.43 |
| K138N/R140E | 0.24 |
| R10E/K14E | 0.47 |
| R10E/K14D | 0.30 |
| R10T/K14D | 0.53 |

The activity of variant TPO proteins with mutations in residues 9-17 are shown in the table below. These variants were selected to have reduced immunogenicity and retain functionally important residues.

| TPO Variant | EC50 |
|---|---|
| L9K/R17K | 0.001072 |
| L9K/R17Q | 4.01E − 05 |
| L9A/V11A/L15A/R17E | 0.2798 |
| L9A/V11A/L15A/R17S | 0.3661 |
| L9A/V11A/K14R/L15A/R17S | 0.5091 |
| L9A/V11A/K14R/L15V/R17E | 5343 |
| L9A/V11I/L15A/R17E | 0.1086 |
| L9A/V11I/L15V/R17E | 0.007998 |
| L9A/V11I/K14R/R17E | 0.001251 |
| L9A/V11I/K14R/L15V/R17E | 0.02322 |
| L9A/L15A/R17E | 0.07736 |
| L9A/R17E | 0.000888 |
| L9A/L15V/R17E | 0.03569 |
| L9A/K14R/L15A/R17E | 0.3481 |
| L9A/K14R/L15V/R17E | 0.07059 |
| L9A | 6.28E − 05 |
| V11A | 0.000741 |
| V11I | 0.3711 |
| K14R | 0.000187 |
| L15A | 0.001617 |
| L15V | 0.000208 |
| R17E | 0.001192 |
| R17K | 0.000133 |
| R17Q | 0.002625 |
| R17S | 0.001565 |
| wt TPO | 6.34E − 05 |

The activity of variant TPO proteins with mutations in residues 129-145 are shown in the table below. These variants were selected to have reduced immunogenicity and retain functionally important residues.

| TPO Variant | EC50 |
|---|---|
| R136K/F141Q/M143L | 0.001741 |
| R136K/V139L/F141Y/M143L | 0.002543 |
| R136K/V139L/F141Q/M143L | 0.007316 |
| L135A/F141Y | 0.02589 |
| L135A/R140K | 0.09264 |
| L135A/R140K/M143L | 0.2729 |
| L135A/R140K/F141H | 5727 |
| L135A/R140K/F141L | 2.644 |
| L135A/R140K/F141L/M143L | 4.728 |
| L135A/R140K/F141Y | 0.01831 |
| L135A/R140K/F141Y/M143L | 0.04429 |
| L144E/V145A | 0.000894 |
| L129E/Q132E/R136K/F141Q/M143L/L144E/V145A | 0.1847 |
| L129E/Q132E/R136K/F141Y/M143L/L144E/V145A | 0.001013 |
| L129E/Q132E/L135A/F141Y/L144E/V145A | 0.001192 |
| L129E/Q132E/L135A/R140A/L144E/V145A | 0.04887 |
| Q132E | 0.000166 |
| L135A | 0.01158 |
| R136K | 5.71E − 05 |
| V139L | 0.001059 |
| R140K | 0.07524 |
| F141H | 0.001178 |
| F141L | 0.001017 |
| F141Q | 0.004986 |
| F141Y | 0.001041 |
| M143L | 6.05E − 05 |
| L144E | 9.72E − 05 |
| WT TPO | 6.34E − 05 |

The activity of variant TPO proteins with mutations in residues 69-77 are shown in the table below. These variants were selected to have reduced immunogenicity and retain functionally important residues.

| TPO Variant | EC50 |
|---|---|
| V74L | 0.001338 |
| M75K | 4.10E − 05 |
| M75Q | 5.10E − 05 |
| V74A | 0.001527 |
| L69A/M75L | 0.000957 |
| L69A/M75Q | 8.32E − 14 |
| L69A | 0.001036 |
| L69Q/M75Q | 0.000123 |
| L69Q | 0.000111 |
| L69A/M75K | 9.93E − 05 |
| L69Q/M75K | 4.51E − 05 |
| L69Q/E72K/M75L | 0.000321 |
| L69Q/E72K | 5.41E − 05 |
| L69A/V74L/M75L | 0.004543 |
| L69Q/E72K/M75K | 0.000142 |
| L69A/V74L | 0.001608 |
| L69Q/V74L | 0.000154 |
| E72K | 0.001963 |
| M75L | 0.001049 |
| wt TPO | 6.34E − 05 |

The activity of variant TPO proteins with mutations in residues 97-105 are shown in the table below. These variants were selected to have reduced immunogenicity and retain functionally important residues.

| TPO Variant | EC50 |
|---|---|
| V97T/R98K/L99I/A103S/Q105E | 0.4953 |
| V97T/R98K/A103S/Q105E | 0.4469 |
| V97T/R98K/L99V/A103S/Q105E | 2.637 |
| V97T/L99I/A103S/Q105E | 0.4178 |
| V97T/A103S/Q105E | 0.8271 |
| V97T/A103S | 0.003357 |
| V97T/L99V/A103S/Q105E | 0.02027 |
| R98K/L100I/Q105E | 0.01138 |
| R98K/L100I | 0.005217 |
| R98K/L99V/L100I/Q105E | 0.09652 |
| R98K/L99V/L100I/A103S/Q105E | 0.06794 |
| R98K/L99V/Q105E | 0.002855 |
| R98K/L99V/A103S/Q105E | 0.001053 |
| R98Q/L99V/Q105E | 0.001117 |
| R98K/L99V | 0.000899 |
| R98Q/L99V/A103S/Q105E | 0.001249 |
| V97T | 2.081 |
| R98K | 0.00027 |
| R98Q | 7.52E − 05 |
| L99I | 0.000236 |
| L99V | 0.000524 |
| L100I | 0.001161 |
| A103S | 0.001222 |
| Q105E | 0.001001 |
| wt TPO | 6.34E − 05 |

1. A non-naturally occurring thrombopoietin (TPO) molecule comprising at least one peptide having reduced binding to at least 1 human MHC Class II allele.

2. A non-naturally occurring TPO molecule according to claim 1, wherein said molecule comprises residues 1-153.

3. A non-naturally occurring TPO molecule according to claim 1, wherein said molecule has at least one amino acid substitution as compared to wild type TPO.

4. A non-naturally occurring TPO molecule of claim 3 wherein said amino acid substitutions are incorporated at one or more of the following positions: 9-24, 69-77, 97-105,128-160, or 296-305.

5. A non-naturally occurring TPO molecule according to claim 4, wherein said amino acid substitutions are incorporated at one or more of the following positions: 9-17.

6. A non-naturally occurring TPO molecule according to claim 4, wherein said amino acid substitutions are incorporated at one or more of the following positions: 129-145.

7. A non-naturally occurring TPO molecule according to claim 4, wherein said amino acid substitutions are incorporated at one or more of the following positions: 69-77.

8. A non-naturally occurring TPO molecule according to claim 4, wherein said amino acid substitutions are selected from amino acid residues at positions 97-105.

9. A non-naturally occurring TPO molecule according to claim 5, wherein said amino acid substitutions are selected from the group consisting of L9E, L9K, L9R, L9S, R10D, R10E, R10S, R10T, K14D, K14E, and K14R.

10. A non-naturally occurring TPO molecule according to claim 9, wherein said amino acid substitutions are selected from the group consisting of L9S, L9K, L9R, L9E, (R10E and K14D), (R10E and K14E), (R10T and K14D).

11. A non-naturally occurring TPO molecule according to claim 5, wherein said amino acid substitutions are selected from the group consisting of: L9A, V11A, V11I, K14R, L15A, L15V, R17K, R17Q, R17S, R17E.

12. A non-naturally occurring TPO molecule according to claim 11, wherein said amino acid substitutions are selected from the group consisting of: (L9A, V11I, K14R, and R17E) or (L9A and R17E).

13. A non-naturally occurring TPO molecule of claim 5 selected from the group consisting of SEQ ID NO.: ______.

14. A non-naturally occurring TPO molecule according to claim 6, wherein said amino acid substitutions are selected from the group consisting of: R136D, R136E, R136K, R136Q, K138N, K138Q, K138R, K138S, K138T, R140D, and R140E.

15. A non-naturally occurring TPO molecule according to claim 14, wherein said amino acid substitution is selected from the group consisting of: R136D, R136E, R136K, R136Q, (K138T and R140E), and (K138N and R140E).

16. A non-naturally occurring TPO molecule according to claim 6, wherein said amino acid substitutions are selected from the group consisting of: L129E, Q132E, L135A, R136K, V139L, R140K, F141Y, F141Q, M143L, L144E, V145A.

17. A non-naturally occurring TPO molecule according to claim 16, wherein said amino acid substitutions are selected from the group consisting of: (L129E, Q132E, R136K, F141Y, M143L, L144E, and V145A) or (L129E, Q132E, L135A, F141Y, L144E, and V145A).

18. A non-naturally occurring TPO molecule according to claim 6 selected from the group consisting of SEQ ID NO.: ______.

19. A non-naturally occurring TPO molecule according to claim 7, wherein said amino acid substitutions are selected from the group consisting of L69A, L69Q, E72K, V74L, M75K, M75L, and M75Q.

20. A non-naturally occurring TPO molecule according to claim 19, wherein said amino acid substitutions are selected from the group consisting of (L69A and M75L), (L69A and M75Q), L69A, (L69Q and M75K), (L69Q, E72K, and M75L), (L69Q, E72K, and M75K), and (L69Q and V74L).

21. A non-naturally occurring TPO molecule according to claim 8, herein said amino acid substitutions are selected from the group consisting of V97T, R98K, R98Q, L99I, L99V, L100I, A103S, and Q105E.

22. A non-naturally occurring TPO molecule according to claim 21, wherein said amino acid substitutions are selected from the group consisting of: (R98K, L99V, and Q105E) and (R98K, L99V, A103S, and Q105E).

23. A non-naturally occurring TPO molecule comprising modifications in at least 2 of the following groups of residues: 9-17, 129-145, 69-77, and 97-105.

24. A non-naturally occurring TPO molecule of claim 1 having at least a 5% reduction in the fraction of patients in which neutralizing antibodies are elicited.

25. A recombinant nucleic acid encoding the non-naturally occurring TPO molecule of claim 1.

26. An expression vector comprising the recombinant nucleic acid of claim 25.

27. A host cell comprising the recombinant nucleic acid of claim 25.

28. A method of producing a non-naturally occurring TPO molecule comprising culturing the host cell of claim 27 under conditions suitable for expression of said nucleic acid.

29. A pharmaceutical composition comprising a non-naturally occurring TPO molecule according to claim 1 and a pharmaceutical carrier.

30. A method for treating a TPO-related disorder comprising administering a non-naturally occurring TPO molecule according to claim 1 to a patient.
